(19) United States
(12) Patent Application Publication
(10) Pub. No.: US 2016/0169893 A1
(43) Pub. Date: Jun. 16, 2016

(54) PCSK9 Function Assay

(71) Applicant: Atherotech, Inc., Birmingham, AL (US)

(72) Inventor: Chen-Hsiung YEH, Birmingham, AL (US)

(21) Appl. No.: 14/958,600

(22) Filed: Dec. 3, 2015

Related U.S. Application Data
(63) Continuation of application No. 14/455,661, filed on Aug. 8, 2014, now abandoned.
Application No. 14/455,661 is a continuation of application No. PCT/US14/49427, filed on Aug. 1, 2014.
(60) Provisional application No. 61/861,126, filed on Aug. 1, 2013.

(51) Int. Cl. G01N 33/573

(52) U.S. Cl. CPC G01N 33/573; G01N 2333/95; G01N 2800/323

(57) ABSTRACT

Methods and apparatuses for measuring the concentration of functional proprotein convertase subtilisin/kexin type 9 (PCSK9). A method of measuring functional PCSK9 in a sample is provided, by contacting the sample with a PCSK9-binding agent capable of binding to the LDL-R-binding region of a PCSK9; and measuring the amount of functional PCSK9 from the sample bound to the binding agent. Diagnostic methods, kits, and reagents for using the method are also provided.

### CROSS-REFERENCE TO RELATED APPLICATIONS

This application is a continuation of U.S. patent application Ser. No. 14/455,661, filed Aug. 8, 2014, which is currently pending. U.S. application Ser. No. 14/455,661 is a continuation under 35 U.S.C. §120 of International Application PCT/US14/49427, filed on Aug. 1, 2014, which is pending. International Application PCT/US14/49427 cited the priority under 35 U.S.C. §119 of provisional U.S. Application 61/861,126, filed on Aug. 1, 2013, which is expired. The contents of International Application PCT/US14/49427 are incorporated herein by reference in their entirety.

### BACKGROUND

A. Field of the Disclosure

The present disclosure relates generally to biochemical assays. More specifically, the present disclosure relates to assays for determining proprotein convertase subtilisin-like/kexin type 9 function. Such assays as well as apparatuses, kits, and methods for use therewith are provided.

B. Background

The analysis of blood lipoproteins is critical in predicting an individual's risk of many chronic diseases, particularly cardiovascular disease such as coronary heart disease (CHD). CHD continues to be the leading cause of death in the United States despite advances made in its diagnosis, treatment, and prevention in recent decades. As per the recently released Heart and Stroke Statistics (2012 Update by the American. Heart Association; Circulation 2012; 125:e2-e220), CHD accounts for 1 in 6 deaths in the US. In 2008 as many as 405,309 people died of CHD and 785,000 were expected to have a new heart attack and another 470,000 people with recurrent attacks. These statistics clearly indicate that prevention of heart disease still remains a formidable task.

Heart disease is a multi-factorial disease and several risk factors such as high blood pressure, smoking, elevated serum low density lipoprotein (LDL) cholesterol, and diabetes are attributed to an increased risk. Among these risk factors, LDL is known to be directly responsible for the build-up of plaque within the arterial wall which results in atherosclerotic disease, which in turn leads to CHD. Lowering LDL cholesterol by pharmacological means or lifestyle changes significantly reduces atherosclerotic disease and CHD.

Cellular uptake and plasma levels of LDL are controlled by the LDL receptor (LDL-R) through binding of circulating LDL to the LDL-R at the cell surface, followed by internalization of the complex by clathrin-mediated endocytosis. In the endosomes, low pH leads to disassociation of the LDL/LDL-R complex, allowing the receptor to recycle to the cell surface while the LDL is degraded in the lysosome. The primary regulator of LDL-R levels and thus of circulating LDL is proprotein convertase subtilisin-like/kexin type 9 (PCSK9). PCSK9 is a serine protease of the proprotein convertase family that regulates circulating LDL-R levels by controlling LDL-R degradation. Since the discovery of the first missense mutation in PCSK9 and the link to an autosomal-dominant form of familial hypercholesterolemia, numerous mutations in the PCSK9 gene have been identified and associated with hypercholesterolemia (gain of function) or hypocholesterolemia (loss of function).

Both PCSK9 and the LDL-R have been well characterized. PCSK9 is a 70 kDa serine protease that contains three domains: an N-terminal prodomain, a subtilisin-like catalytic domain, and a C-terminal cysteine/histidine-rich domain (CTD). PCSK9 undergoes autocatalytic cleavage, but the 14 kDa prodomain remains noncovalently attached to the catalytic domain and renders the protease inactive. It is believed that the prodomain acts as a chaperone and assists in folding of the protein, whereas autocatalytic processing is crucial for the secretion of PCSK9. The LDL-R is a multidomain protein that comprises (1) an extracellular domain with an N-terminal ligand-binding domain that includes seven cysteine-rich repeats (L1-L7), (2) two epidermal growth factor (EGF) homology domains (EGF-A and EGF-B) that are separated from a third EGF-like domain (EGF-C) by a β-propeller domain, and (3) an “O-linked sugar” domain.

Functional studies have shown that the plasma levels of LDL are regulated by PCSK9 through inhibition of the recycling of LDL-R to the surface following internalization, leading to degradation of the LDL-R in the liver. Absent the presence of PCSK9, the LDL-R recycles to the cell membrane for further internalization of circulating LDL. The two proteins interact via a 530 A°² flat contact patch between the catalytic domain of PCSK9 and the EGF-A domain in the LDL-R. The interface involves a central hydrophobic patch with a number of surrounding polar interactions and putative salt bridges contributing to the high binding specificity.

The discovery of the role of PCSK9 in LDL metabolism has led to studies as to its role as a biomarker and therapeutic target in the cardiovascular field. However, the effective measurement of PCSK9 has remained elusive. This is because PCSK9 is only able to bind the LDL-R if it is not already bound to another molecule. Such unbound PCSK9 that is available to bind to LDL-R is referred to as “functional PCSK9,” and it is the form that is of clinical significance to CHD and other disease states.

So far, only a few methods (mainly antibody-based) for the detection of total steady-state PCSK9 levels in circulation have been reported. These are universally ineffective for indicating a patient's risk of CHD based on PCSK9 levels because they are incapable of distinguishing between functional PCSK9 and non-functional PCSK9. Consequently, there is an unmet need in the art for a way to measure functional PCSK9.

### SUMMARY

The following presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The disclosure provides a method of measuring functional PCSK9 in a sample, the method comprising: contacting the sample with a PCSK9-binding agent that binds the LDL-R-binding region of said functional PCSK9 for a period sufficient to allow substantially all of the PCSK9 in the sample to bind to the binding agent; and measuring directly or indirectly the amount of functional PCSK9 from the sample bound to the binding agent.

A method of evaluating the risk of atherosclerotic disease in a subject is also provided, the method comprising: performing the above method of measuring functional PCSK9 on a sample from the subject; and determining the subject's risk of atherosclerotic disease based on the amount of functional PCSK9 detected.

The disclosure provides an apparatus for measuring functional PCSK9 in a sample, the apparatus comprising: a substrate with low binding affinity to PCSK9; and a PCSK9-binding agent associated with the substrate that is capable of binding to an LDL-R-binding region of a PCSK9. The disclosure also provides a kit comprising the same apparatus, and comprising a signal compound, the signal compound capable of binding to the binding agents, the signal compound comprising a reporter.

The disclosure provides a fluorescence resonance energy transfer (FRET) reagent for the detection of functional PCSK9 comprising a PCSK9-binding agent conjugated to a first fluorophore. Methods of using the FRET reagent and kits containing the FRET reagent are also provided.

### BRIEF DESCRIPTION OF THE DRAWINGS

FIG. 1: An alignment of PCSK9 sequences of three primate species. In this figure SEQ ID NO: 12=Homo sapiens PCSK9, SEQ ID NO: 17=Pan troglodytes PCSK9, and SEQ ID NO: 18=Macaca mulatta PCSK9

FIG. 2: A consensus sequence of PCSK9 from five mammal species showing the common properties of non-identical amino acids.

FIG. 3: The consensus sequence of PCSK9 from five mammal species shown in FIG. 2 allowing any substitution for non-identical amino acids.

FIG. 4: An alignment of PCKS9 from five mammal species.

FIG. 5: A consensus sequence of the PCSK9-binding domain of LDRL from five mammal species showing the common properties of non-identical amino acids.

FIG. 6: Exemplary standard curve of ABS₄₅₀ plotted against concentration of free (functional) PCSK9 using seven-point standard solutions.

FIG. 7: Additional standard curve of ABS₄₅₀ plotted against concentration of free (functional) PCSK9 using seven-point standard solutions.

FIG. 8: Exemplary standard curve of ABS₄₅₀ plotted against concentration of total PCSK9 using seven-point standard solutions.

FIG. 9: Additional standard curve of ABS₄₅₀ plotted against concentration of total PCSK9 using seven-point standard solutions.

FIG. 10: Linearity of 3 pooled samples (low, medium, and high) of measured total PCKS9.

FIG. 11: Linearity of 3 pooled samples (low, medium, and high) of measured free (functional) PCKS9.

FIG. 12: An embodiment of the apparatus, comprising the substrate (100) and the PCSK9-binding agent (200); and showing PCSK9 from the sample (300), the signal compound (400) conjugated to the reporter (500).

### DETAILED DESCRIPTION

### A. Definitions

With reference to the use of the word(s) “comprise” or “comprises” or “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “consisting essentially of” means that, in addition to the recited elements, what is claimed may also contain other elements (steps, structures, ingredients, components, etc.) that do not adversely affect the operability of what is claimed for its intended purpose.

Articles such as “the” and “a” are not intended to limit a given element or step to only a single one of its type, and it is to be understand that when reference is made to “an element” or “a step” that more than one such element or step may be present unless specified to the contrary. Likewise, it is to be understand that when reference is made to “the element” or “the step” that more than one such element or step may be present unless specified to the contrary. Such articles should generally be read to refer to “at least one” element or step.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given herein are approximate unless stated otherwise, meaning that the term “about” or “approximately” can be inferred when not expressly stated.

The term “individual,” “subject,” or “patient” as used herein refers to any animal, including mammals, such as mice, rats, other rodents, rabbits, dogs, cats, swine, cattle, sheep, horses, primates, and humans. The term may specify male or female or both, or exclude male or female.

### B. Methods of Measuring Functional PCSK9

Methods of measuring functional PCSK9 in a sample are provided. A general embodiment of the method comprises: contacting a sample with a PCSK9-binding agent that binds the LDL-R-binding region of said functional PCSK9 for a period sufficient to allow substantially all of the PCSK9 in the sample to bind to the PCSK9 binding agent; contacting the PCSK9 binding agent with a signal compound, the signal compound comprising a LDL-R-binding region capable of binding to the PCSK9 binding agent, and a reporter; and measuring the amount of signal compound bound to the PCSK9 binding agent, wherein the amount of signal compound bound to the PCSK9 binding agent is inversely related to the amount of functional PCSK9 in the sample. Some embodiments of the method further comprise removing any unbound signal compound. Some embodiments of the method further comprise obtaining the sample from a subject. Some embodiments of the method are performed ex vivo (for example, in vitro).

In this context, “functional” PCSK9 refers to PCSK9 that is capable of binding the LDL-R and available to do so. The functional PCSK9 may or may not have other functions associated with that molecule for the purposes of this disclosure.

Some embodiments of the PCSK9-binding agent comprise a PCSK9-binding region. In some embodiments of the method more than a single PCSK9-binding region will be present. In such embodiments the PCSK9-binding regions used need not be identical to one another, although they may. It is contemplated that two or more PCSK9-binding regions could be part of the same molecule, such that the more than two PCSK9-binding regions are positioned to be in contact with the sample. A specific embodiment of the PCSK9-binding agent comprises a PCSK9-binding region of a LDL-R.

Unbound signal compound may be removed by any suitable separation method. In some embodiments of the method the binding agent may be immobilized to a substrate, in which case unbound signal compound may be removed by washing the substrate. In such embodiments it may be sufficient merely to drain any liquid carrying the signal compound. In other embodiments of the method the signal compound may be removed by degrading it through chemical reaction (for example to neutralize the reporter). In further embodiments the reporter may be neutralized selectively in signal compound molecules that have not bound to the PCSK9-binding agent.

The amount of signal compound present may be measured by quantitative detection of the reporter. For example, if the reporter is a fluorescent moiety, the signal compound may be measured by exciting the fluorescent moiety and measuring the fluorescence. If the reporter is a radionuclide, the signal compound may be measured by radiometry. If the reporter is an enzyme, the signal compound may be measured by exposing the reporter to the enzyme's substrate and measuring enzyme activity (i.e., measuring the rate of substrate consumption or the rate of product generation). If the reporter is a dye then the signal compound may be measured colorimetrically. If the reporter is a magnetic particle then the signal compound may be measured by introducing it to a magnetic field and observing the migration of the complex of the signal compound and PCSK9-binding agent.

If the reporter is a donor fluorophore, then the signal compound may be measured by the level of emission from an acceptor fluorophore that is associated with the binding agent (for example, in methods of fluorescence resonance energy transfer (FRET)). If the reporter is an acceptor fluorophore, then the signal compound may be measured by the level of emission from the acceptor fluorophore (in such embodiments a donor fluorophore will be associated with the binding agent). Put another way, the signal compound may be conjugated to a fluorophore, and the reporter in the signal compound would be a “complementary” fluorophore. In this context a complementary fluorophore is a donor fluorophore capable of electron transfer to stimulate the emission of a given acceptor fluorophore; or an acceptor fluorophore capable of emission in response to electron transfer from a given donor fluorophore.

Of course, LDL binds to LDL-R. While not wishing to be bound to any given hypothetical model, it is not believed that LDL in a sample will interfere with the method, due to the fact that the PCSK9-binding site and the LDL-binding site are separate domains that are distant from one another. However, under some circumstances it may be desirable to reduce or eliminate the levels of LDL in the sample. Consequently, some embodiments of the method comprise removing LDL from the sample. More specifically, some embodiments of the method comprise removing free LDL from the sample (LDL that is not already bound to LDL-R or other LDL-binding molecules). Alternatively, the method may comprise removing all (or substantially all) of the LDL from the sample. Such removal may be achieved by any conventional method. Some embodiments of the method comprise removing the LDL from the sample by centrifugation. A specific embodiment of the method comprises removing the LDL from the same by density-gradient centrifugation. Removal of LDL may be achieved by other means such as filtration, extraction, immunoprecipitation, etc. The removal need not be absolute to be useful, and traces of LDL may remain in the sample in some embodiments of the method.

Due to the expected lack of interference by LDL, in some embodiments of the method the sample contains LDL (although a sample from which LDL has been reduced or eliminated can also be used). Samples containing LDL have the advantage of less preparation, for example in the case of blood samples.

In some embodiments of the method an excess of binding agent is present compared to the expected PCSK9 in the sample. Put another way, the number of available binding sites for PCSK9 on the PCSK9-binding agent provided will exceed the expected number of functional PCSK9 molecules in the sample. In some embodiments this translates to the presence of a number of binding agent molecules that exceeds the number of PCSK9 molecules expected in the sample. In other embodiments the binding agent might have multiple binding sites, in which case there might still be an “excess of binding peptide” compared to expected PCSK9 in the same, even if the number of binding agent molecules is smaller than the number of expected PCSK9 molecules.

The sample may be any that is suspected to contain functional PCSK9. For example, the sample may be whole blood or a blood fraction. Examples of such blood fractions include serum, plasma, and sub-fractions thereof. As PCSK9 is usually found in the serum fraction of the subject's blood, the blood fraction will generally not be a fraction from which the serum fraction has been eliminated.

In some instances it may be useful to compare a subject's functional PCSK9 to a measure of the subject's total PCSK9. In such instances the method may further comprise measuring the total PCSK9 in the sample. The total PCSK9 may be compared to the functional PCSK9 to calculate a ratio. The ratio may be calculated on any suitable basis, such as by stoichiometry, based on mass-concentration, or based on total mass in the sample. The subject's total PCSK9 may be measured in the sample, or by another suitable means. A specific embodiment of the method comprises measuring the total circulating concentration of PCSK9 in the subject (wherein the original sample was from the subject).

PCSK9-Binding Regions

In some embodiments of the method, the PCSK9-binding regions used in this disclosure may be polypeptides comprising at least a PCSK9-binding domain, a functional derivative of a PCSK9-binding domain or a fragment of either of the foregoing. In one embodiment, the PCSK9-binding domain is a PCSK9-binding domain from an LDL-R. The PCSK9-binding region, derivative, or fragment has the property of binding to PCSK9.

When the PCSK9-binding domain is from an LDL-R, the LDL-R from which the binding region is derived may be any isoform or from any species. Some embodiments of the LDL-R are a human LDL-R, such as isoform 1 (SEQ ID NO: 1). In other embodiments the LDL-R may be human isoform 2 (SEQ ID NO: 4), isoform 3 (SEQ ID NO: 5), isoform 4 (SEQ ID NO: 6), or isoform 5 (SEQ ID NO: 7). In still further embodiments the LDL-R may be from another mammalian species, such as Pan troglodytes (SEQ ID NO: 2), Macaca mulatta (SEQ ID NO: 3), Mus musculus (SEQ ID NO: 15), or Rattus norvegicus (SEQ ID NO: 16).

The EGF-AB domain of LDL-R has been shown to bind PCSK9. This domain resides at positions 314-393 of SEQ ID NO: 1 in the case of the canonical human LDL-R isoform 1. The EGF-A domain, which resides at positions 314-353 of SEQ ID NO: 1 in the case of the canonical human LDL-R isoform 1, is believed to be capable of binding PCSK9 alone. The PCSK9-binding regions used in this disclosure may comprise either of the EGF-AB or EGF-A domains. Furthermore, fragments of the EGF-AB or EGF-A domain may also be used. In such embodiments the fragments may be a fragment comprising the residues at positions 314-393 of SEQ ID NO: 1 or positions 314-353 of SEQ ID NO:1, the corresponding fragment from a human LDL-R isoform other than isoform 1, or the corresponding fragment from a non-human LDL-R.

The PCSK9-binding region may be an N-terminal region of the EGF-A domain. The N-terminal region of the EGF-A domain has been observed to display binding activity with LDL-R. This N-terminal region comprises 26 residues on the N-terminal end of the EGF-A, found at positions 1-26 of the PCSK9 binding domain (positions 1-26 of SEQ ID NOS: 8-11, or positions 314-339 of SEQ ID NOS: 1-7, 15, 16, and 24-25). Correspondingly, the PCSK-9 binding region may comprise a sequence selected from positions 1-26 of any one of SEQ ID NOS: 8-11 and/or positions 314-339 of any one of SEQ ID NOS: 1-7, 15, 16, and 24-25.

The PCSK9-binding region may also depart from an established canonical sequence to account for observed natural variants. For example, human isoform 1 of LDL-R is known to contain natural variants in at least 18 locations at positions 314-393 (the EGF-AB domain), 9 of which are located at positions 314-353 (the EGF-A domain), and 8 of which are located at positions 314-339 (the N-terminal region of the EGF-A domain). Some such known natural variants are listed here in Table 1.

TABLE 1
Natural Variants EGF-AB Domain of Human LDL-R

| Position | Substitution |
|---|---|
| 318 | C → F |
| 318 | C → R |
| 318 | C → Y |
| 327 | H → Y |
| 329 | C → F |
| 329 | C → Y |
| 335 | G → S |
| 338 | C → S |
| 342 | D → E |
| 342 | D → N |
| 343 | G → S |
| 350 | R → P |
| 352 | C → Y |
| 354 | D → G |
| 354 | D → V |
| 356 | D → Y |
| 357 | E → K |
| 358 | C → Y |
| 364 | C → R |
| 366 | Q → R |
| 368 | C → R |
| 370 | N → T |
| 379 | C → R |
| 379 | C → Y |
| 391 | A → T |

For example, in some embodiments the PCSK9-binding region is SEQ ID NO: 8 (positions 314-353 of human isoform 1 LDRL including the possible substitutions from Table 1). In another example, the PCSK9-binding region is SEQ ID NO: 9 (positions 314-393 of human isoform 1 LDRL including the possible substitutions from Table 1). In a further example, the PCSK9-binding region is SEQ ID NO: 24 (positions 314-353 of human isoform 1 LDRL allowing for any substitution at the positions shown in Table 1). In a further example, the PCSK9-binding region is SEQ ID NO: 25 (positions 314-393 of human isoform 1 LDRL allowing for any substitution at the positions shown in Table 1). In one embodiment, the PCSK9-binding region is a sequence as shown in any one of SEQ ID NOS: 1-11, 15, 16, 24, and 25 that includes a tyrosine for histidine substitution at the position corresponding to position 327 in SEQ ID NO: 1. In further embodiments, the PCSK9-binding region is the N-terminal region of the EGF-A domain of any of the above sequences.

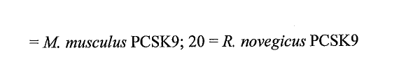

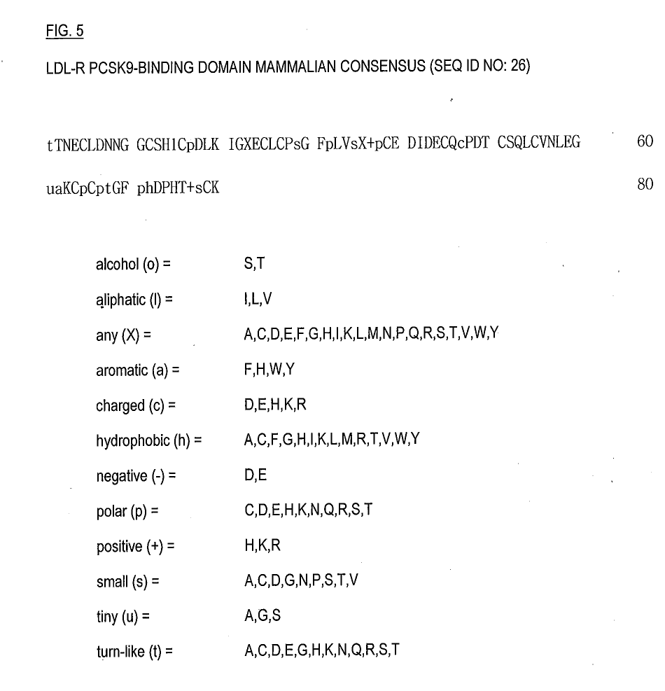

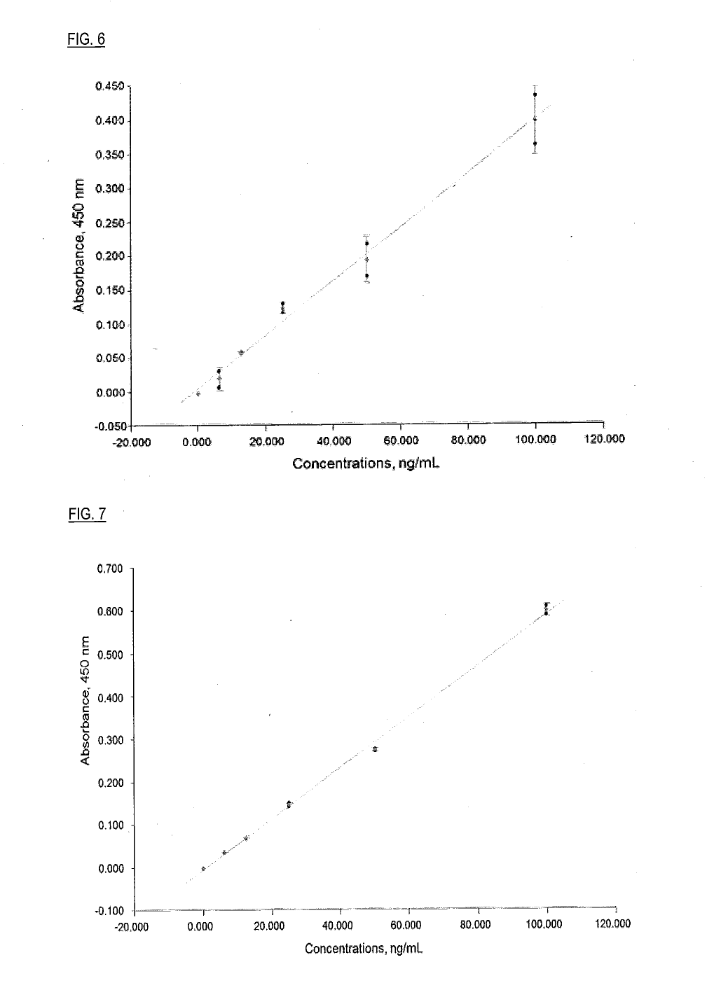

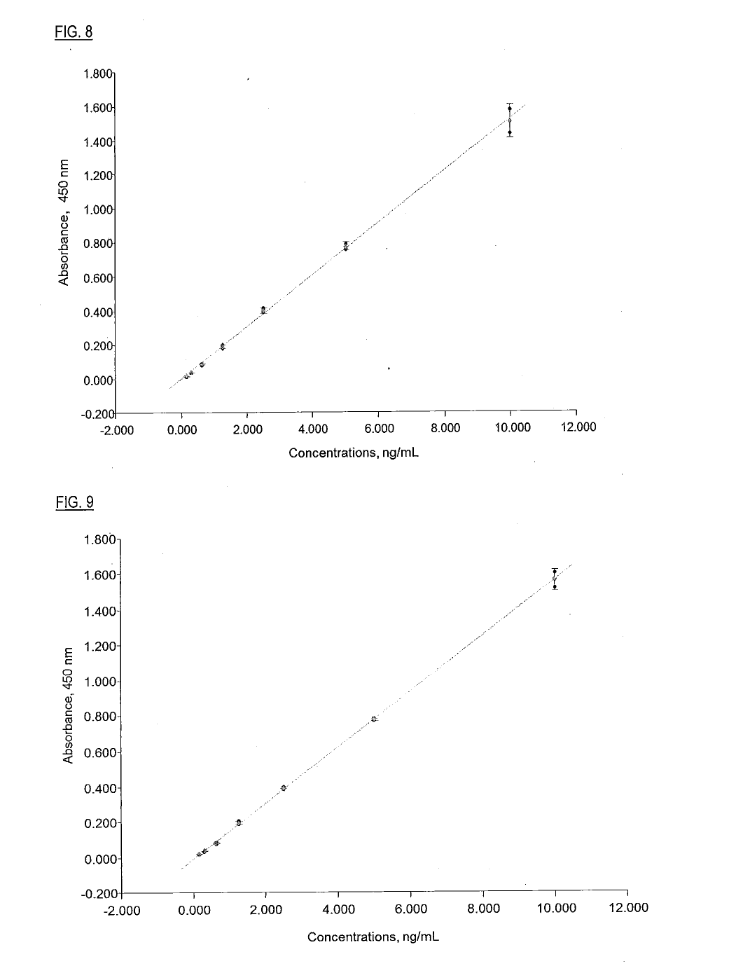

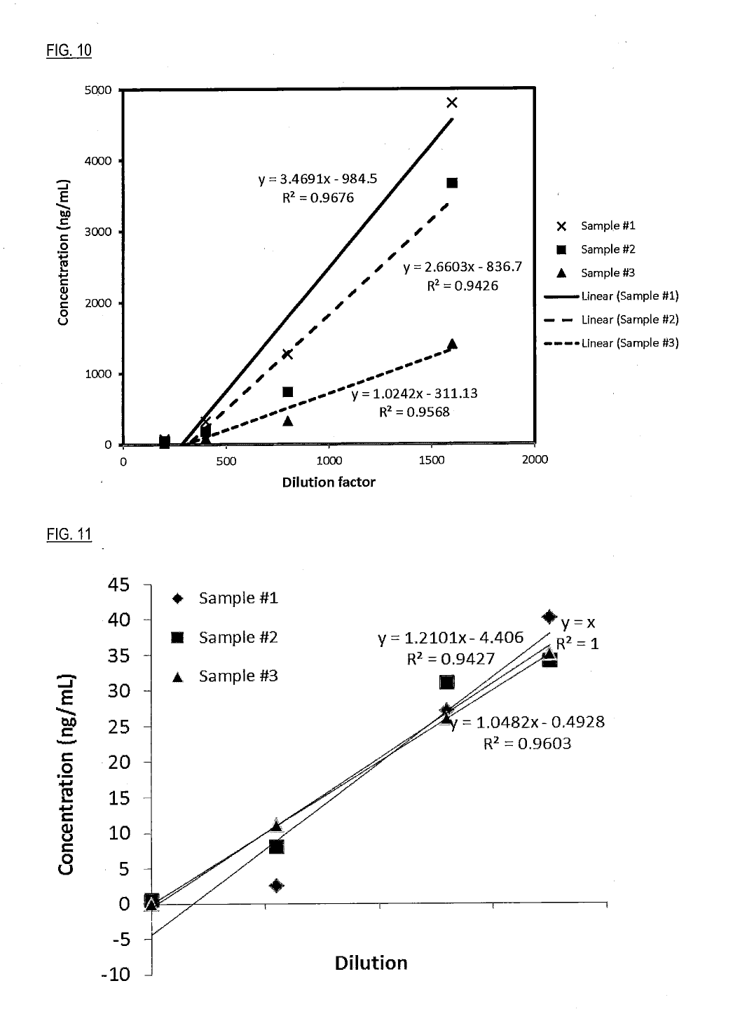

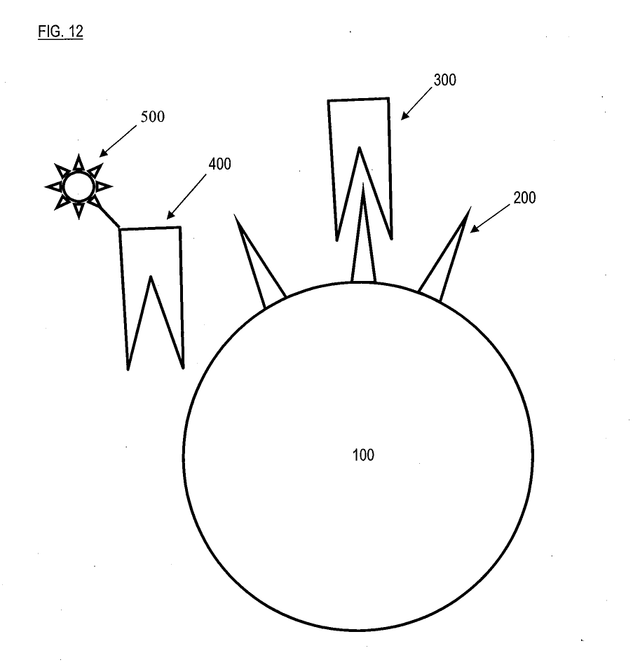

The PCSK9-binding region may also be a consensus sequence from multiple species of the domain that corresponds to the EGF-AB domain, the EGF-A domain, or the N-terminal region of the EGF-A domain of human LDL-R. As is well known in the art, when peptides from multiple species have the same function, one of ordinary skill in the art can reasonably assume that portions of the molecule that are not conserved between species may be varied without eliminating function. One embodiment of the PCSK9-binding region is SEQ ID NO: 10, which is the consensus sequence between human isoform 1 of LDL-R at positions 314-393, Pan troglodytes LDL-R at positions 314-393, and the corresponding region of Macaca mulatta LDL-R. Another embodiment of the PCSK9-binding region comprises positions 1-40 of SEQ ID NO: 10 (the consensus sequence between the catalytic domain of human isoform 1 of LDL-R, Pan troglodytes LDL-R, and the corresponding region of Macaca mulatta LDL-R). Another embodiment of the PCSK9-binding region comprises SEQ ID NO: 11, which is the consensus sequence between human isoform 1 of LDL-R at positions 314-393 and the corresponding regions of LDL-R in Pan troglodytes, Macaca mulatta, Rattus norvegicus, and Mus musculus. Another embodiment of the PCSK9-binding region comprises positions 1-40 of SEQ ID NO: 11LDL-RLDL-R. A more careful analysis of the consensus sequence between human isoform 1 of LDL-R at positions 314-393 and the corresponding regions of LDL-R in the four non-human species is presented in FIG. 5 (SEQ ID NO: 26), and accordingly a particular embodiment of the PCSK9-binding region is the peptide sequence shown in SEQ ID NO: 26. A further particular embodiment of the PCSK9-binding region is positions 1-26 of SEQ ID NO: 26.

The binding agent may also comprise functional derivatives of any of the foregoing sequences, as further described below.

LDL-R-Binding Regions

The LDL-R-binding regions used in the signal compounds are polypeptides that may comprise at least the LDL-R-binding region of a PCSK9, a functional derivative of the LDL-R-binding region of a PCSK9, or a fragment of either of the foregoing. The LDL-R-binding region, derivative, or fragment, has the property of binding to the binding agent.

The PCSK9 from which the binding region is derived may be any isoform from any species. For example, the PCSK9 may be human PCSK9, such as the canonical human isoform 1 (SEQ ID NO: 12). More specifically, the LDL-R-binding region may be the catalytic domain of PCSK9, or a fragment thereof. In a specific embodiment the LDL-R-binding region is the catalytic domain of the canonical human isoform 1 of PCSK9, found at positions 152-452 (SEQ ID NO: 13). The LDL-R-binding region may be a fragment of the catalytic domain that departs from an established canonical sequence to account for observed natural variants. For example, human isoform 1 of PCSK9 is known to contain natural variants in at least 16 locations in the catalytic domain. Some such known natural variants are listed here in Table 2.

TABLE 2
Natural Variants of Catalytic Domain of Human PCSK9

| Position | Substitution |
|---|---|
| 157 | N→K |
| 174 | P→S |
| 215 | R→H |
| 216 | F→L |
| 218 | R→S |
| 219 | Q→E |
| 237 | R→W |
| 239 | A→D |
| 253 | L→F |
| 357 | R→H |
| 374 | D→Y |
| 374 | D→Y |
| 391 | H→N |
| 394 | G→S |
| 417 | H→Q |
| 425 | N→S |
| 443 | A→T |
| 452 | G→D |

For example, some embodiments of the LDL-R-binding region are SEQ ID NO: 14 (positions 152-452 of human PCSK9 isoform 1 including the possible substitutions in Table 2).

The PCSK9 from which the binding region is derived may be from a non-human species. Specific examples include Pan troglodytes (SEQ ID NO: 17), Macaca mulatta (SEQ ID NO: 18), Mus musculus (SEQ ID NO: 19), and Rattus norvegicus (SEQ ID NO: 20).

The LDL-R-binding region may also be a consensus sequence from multiple species of the domain that corresponds to the catalytic domain of human PCSK9. As is well known in the art, when peptides from multiple species have the same function, one of ordinary skill in the art can reasonably assume that portions of the molecule that are not conserved between species may be varied without eliminating function.

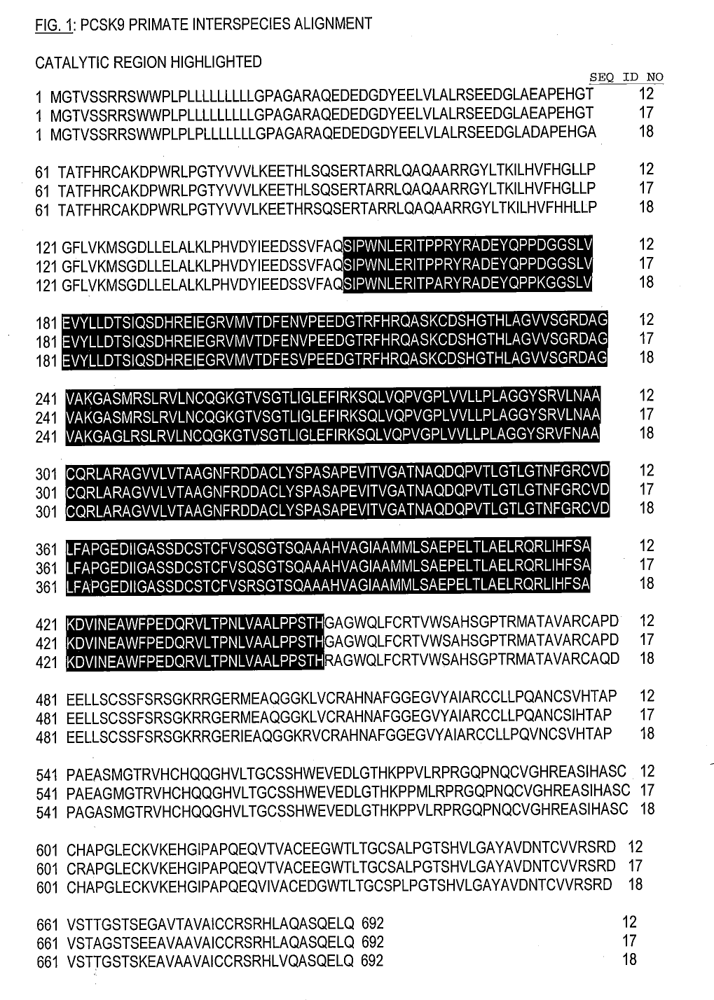

For example, comparing the canonical sequences of PCSK9 in Homo sapiens, Pan troglodytes, and Macaca mulatta reveals complete identity except for 30 amino acids (FIG. 1). As such, some embodiments of the LDL-R-binding region comprise positions 152-452 of the consensus sequence between these three primate species (SEQ ID NO: 21). More specific embodiments of the LDL-R-binding region comprise SEQ ID NO: 21. In further embodiments of the LDL-R-binding region comprising the primate consensus sequence, the amino acids at the 30 non-identical locations are chosen from the following table:

TABLE 3
Amino Acid Selections for Primate Consensus PCSK9

| Position | Selection |
|---|---|
| 16 | L or P |
| 54 | E or D |
| 60 | T or A |
| 88 | L or R |
| 117 | G or H |
| 164 | P or A |
| 175 | D or K |
| 207 | N or S |
| 246 | S or G |
| 247 | M or L |
| 297 | L or F |
| 382 | Q or R |
| 450 | G or R |
| 479 | P or Q |
| 500 | M or I |
| 507 | L or R |
| 532 | A or V |
| 536 | V or I |
| 543 | E or G |
| 545 | S or G |
| 578 | V or M |
| 602 | H or R |
| 623 | T or I |
| 628 | E or D |
| 637 | A or P |
| 664 | T or A |
| 669 | E or K |
| 670 | G or E |
| 673 | T or A |
| 685 | A or V |

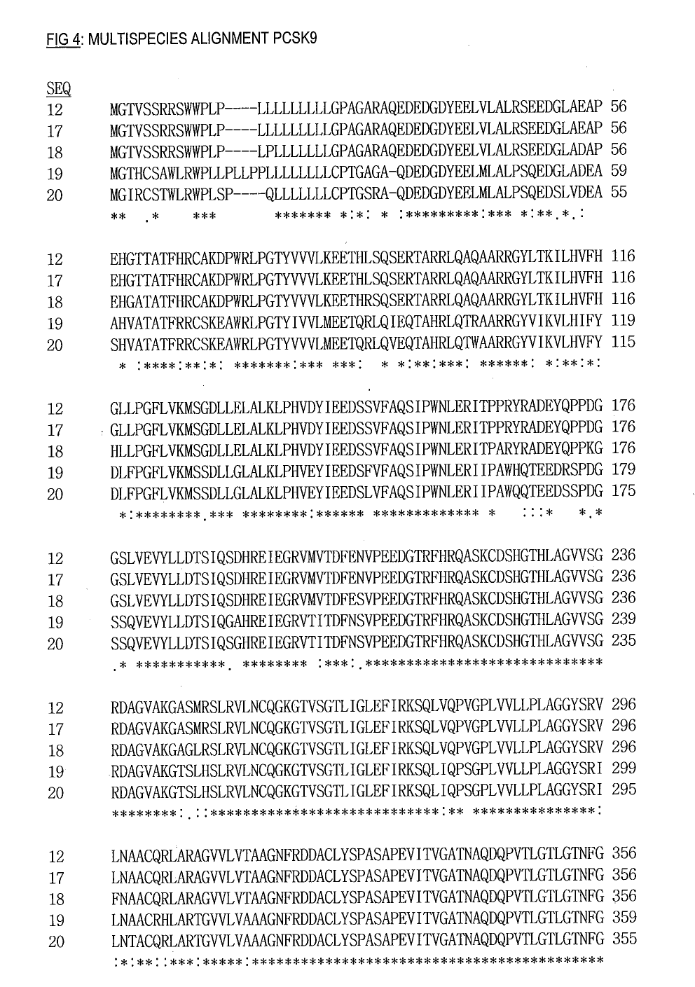

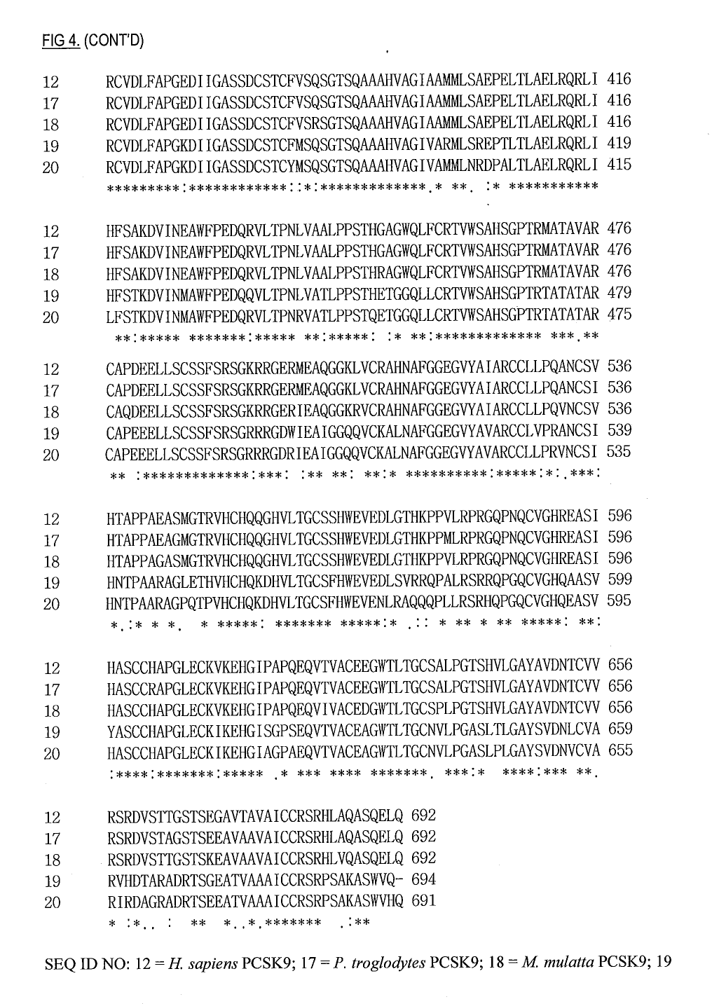

In another example, comparing the canonical sequences of PCSK9 in Homo sapiens, Pan troglodytes, Macaca mulatta, Mus musculus, and Rattus norvegicus reveals complete identity at 508 loci, and differences at the remaining 184 (FIG. 4). As such, some embodiments of the LDL-R-binding region comprise positions 152-452 of the consensus sequence between these mammal species (SEQ ID NO: 22 is the consensus sequence of the catalytic domain; SEQ ID NO: 21 is the consensus sequence of PCSK9, wherein a 0-5 residue spacer sequence may be present between positions 14 and 15, and wherein a 0-1 residue spacer sequence may be present between positions 61 and 62). Such versions of the LDL-R-binding region may be embodied by the following structure:

R₁-R₂-R₃-R₄-R₅

in which:
R₁ is a sequence with a minimum level of homology to SEQ ID NO: 27, said minimum level of homology selected from: 75, 80, 85, 90, 95, 97, 98, 99, 99.5, and 100%;
R₂ is an oligopeptide of 0-5 residues;
R₃ is a sequence with a minimum level of homology to SEQ ID NO: 28, said minimum level of homology selected from: 75, 80, 85, 90, 95, 97, 98, 99, 99.5, and 100%;
R₄ is a peptide of 0-1 residues; and
R₅ is a sequence with a minimum level of homology to positions 31-691 SEQ ID NO: 22 limited to variants in which each of positions 31, 47, 86, 92, 166, 171, 205, 301, 381, 433, 448, 505, 530, 554, 573, 574, 591, 662, 665, and 685 are independently selected from C, D, E, H, K, N, Q, R, S, or T; each of positions 41, 82, 87, 108, 118, 161, 164, 200, 246, 296, 379, 407, 416, 439, 452, 455, 469, 498, 499, 511, 577, 622, 642, and 652, are independently selected from A, C, F, G, H, I, K, L, M, R, T, V, W, or Y; each of positions 50, 67, 127, 176, 190, 245, 544, 616, 648, and 661 are independently selected from A, G, or S; each of positions 52, 55, 58, 59, 70, 99, 163, 167, 172, 191, 206, 244, 279, 298, 306, 312, 395, 400, 419, 442, 450, 473, 531, 537, 538, 540, 555, 569, 572, 580, 585, 615, 635, 636, 640, 643, 655, 660, 663, 664, 671, 672, 674, and 684 are independently selected from A, C, D, G, N, P, S, T, and V; each of positions 53, 69, 140, 168, 402, 479, and 497 are independently selected from D or E; each of 54, 56, 116, 131, 401, 404, 449, 542, 546, 571, 582, 592, 618, 627, 668, and 669 are independently selected from A, C, D, E, G, H, K, N, Q, R, S, or T; each of positions 64, 95, 247, 302, 493, 509, 601, and 658 are independently selected from H, K, or R; each of positions 78, 107, 110, 113, 201, 276, 295, 522, 428, 535, 595, and 609 are independently selected from I, L, or V; each of positions 115, 378, and 596 are independently selected from F, H, W, or Y; each of positions 174 and 365 are independently selected from D, E, H, K, or R; and said minimum level of homology selected from: 75, 80, 85, 90, 95, 97, 98, 99, 99.5, and 100%.

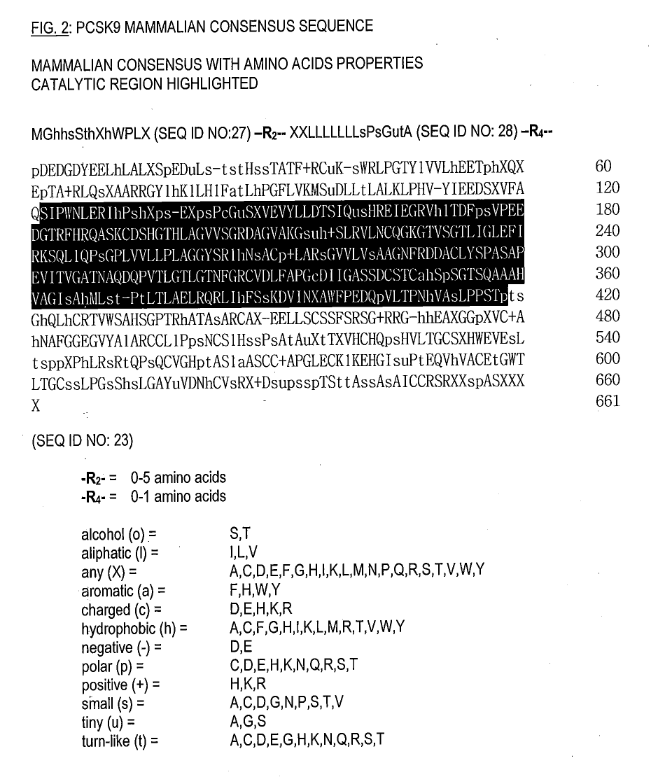

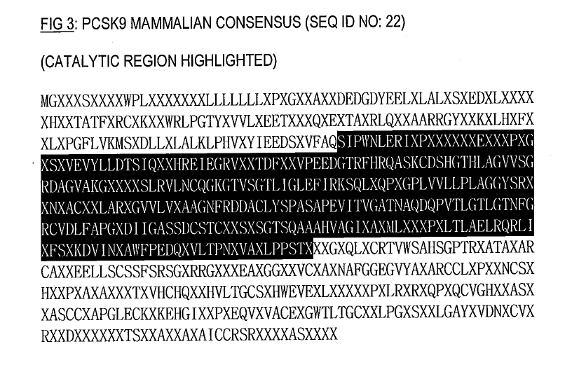

FIGS. 2-3 show the multispecies consensus sequence of PCSK9 in more detail.

The LDL-R-binding region may also comprise functional derivatives of any of the foregoing sequences, as further described below.

Functional Derivatives of the PCSK9-Binding Region and LDL-R-Binding Region

The present disclosure contemplates the use of functional derivatives of the PCSK9-binding region and LDL-R-binding region in the methods disclosed herein. A “derivative” as defined herein refers to a functional PCSK9-binding region or LDL-R-binding region polypeptide that includes one or more fragments, insertions, deletions, or substitutions. The derivative may have an activity that is comparable to or increased (in one embodiment, 50% or more) as compared to the wild-type activity and as such may be used to increase activity; alternatively, the derivative may have activity that is decreased (in one embodiment, less than 50%) as compared to the wild-type activity and as such may be used to decrease activity. In some cases the derivative will retain antigenic specificity of the native peptide.

A fragment is any polypeptide consisting of any number of adjacent amino acid residues having the same identity and order as any segment of the original. Conservative modifications to the amino acid sequence of any fragment are also included (conservative substitutions are discussed below). Such fragments can be produced for example by digestion with an endoprotease (which will produce two or more fragments) or a synthetic exoprotease; such fragments may also be produced via chemical peptide synthesis. A fragment may be of any length up to the total length of the native polypeptide. A fragment may be, for example, at least 3 residues in length. A fragment that is at least 6 residues in length will generally function as an antigenic group. Such groups would be expected by those of ordinary skill in the art to be cross-recognized by some antibodies. Fragments that are homologous to parts of the binding region would be expected to retain binding activity.

Derivatives will have some degree of homology with the native polypeptide. For example, those skilled in the art would expect that most derivatives having from 95-100% homology with native polypeptide would retain its function. It is also within the abilities of those skilled in the art to predict the likelihood that functionality would be retained by a homolog within any one of the following ranges of homology: 75-100%, 80-100%, 85-100%, and 90-100%. Persons having ordinary skill in the art will understand that the minimum desirable homology can be determined in some cases by identifying a known non-functional homolog, and establishing that the minimum desirable homology must be above the known non-functional homology level. Persons having ordinary skill in the art will also understand that the minimum desirable homology can be determined in some cases by identifying a known functional homolog, and establishing that the range of desirable homology may be equal to or greater than about the homology level of the known functional homolog.

Deletions, additions and substitutions can be selected, as would be known to one of ordinary skill in the art, to generate a desired derivative. For example, it is not expected that deletions, additions and substitutions outside of the binding regions of the polypeptide would alter binding activity. Likewise, conservative substitutions or substitutions of amino acids with similar properties is expected to be tolerated in the binding region, and binding activity may be conserved. Of course non-conservative substitutions in these regions would be expected to decrease or eliminate a binding activity.

Conservative modifications to the amino acid sequence of the binding region (and the corresponding modifications to the encoding nucleotides) will produce derivatives having functional and chemical characteristics similar to those occurring naturally. In contrast, substantial modifications in functional and/or chemical characteristics may be accomplished by selecting substitutions in the amino acid sequence of the binding region that differ significantly in their effect on maintaining (a) the structure of the molecular backbone in the area of the substitution, (b) the charge or hydrophobicity of the molecule at the binding site for a binding target, or (c) the bulk of a side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine.

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties. It will be appreciated by those of skill in the art that nucleic acid and polypeptide molecules described herein may be chemically synthesized as well as produced by recombinant means.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

For example, non-conservative substitutions may involve the exchange of a member of one of these classes for a member from another class.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (Kyte et al., J. Mol. Biol., 157:105-131, 1982). It is known that certain amino acids may be substituted for other amino acids having a similar hydropathic index or score and still retain a similar biological activity.

In making changes based upon the hydropathic index, the substitution of amino acids whose hydropathic indices are within +/−2 may be used; in an alternate embodiment, the hydropathic indices are within +/−1; in yet another alternate embodiment, the hydropathic indices are within +/−0.5.

It is also understood in the art that the substitution of like amino acids can be made effectively on the basis of hydrophilicity. The greatest local average hydrophilicity of a polypeptide as governed by the hydrophilicity of its adjacent amino acids, correlates with a biological property of the protein.

The following hydrophilicity values have been assigned to amino acid residues: arginine (+3.0); lysine (+3.0); aspartate (+3.0.±0.1); glutamate (+3.0.±0.1); serine (+0.3); asparagine (+0.2); glutamine (+0.2); glycine (0); threonine (−0.4); proline (−0.5.±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within +/−2 may be used; in an alternate embodiment, the hydrophilicity values are within +/−1; in yet another alternate embodiment, the hydrophilicity values are within +/−0.5.

Desired amino acid substitutions (whether conservative or non-conservative) can be determined by those skilled in the art at the time such substitutions are desired. For example, amino acid substitutions can be used to identify important residues of the binding region.

Exemplary amino acid substitutions are set forth in Table 4.

TABLE 4
Conservative Substitutions

| Original | | Preferred |
|---|---|---|
| Amino Acid | Exemplary substitution | substitution |
| Ala | Val, Leu, Ile | Val |
| Arg | Lys, Gln, Asn | Lys |
| Asn | Glu | Glu |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Ile, Val, Met, Ala, Phe, Norleucine | Ile |
| Lys | Arg, 1,4-diaminobutyric acid, Gln, Asn | Arg |
| Met | Leu, Phe, Ile | Leu |
| Phe | Leu, Val, Ile, Ala, Tyr | Leu |
| Pro | Ala, Gly | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

A skilled artisan will be able to determine suitable variants of the polypeptide as set forth in the sections above, including combinations thereof, using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of the polypeptide to such similar polypeptides. With such a comparison, one can identify residues and portions of the molecules that are conserved among similar polypeptides. It will be appreciated that changes in areas that are not conserved relative to such similar polypeptides would be less likely to adversely affect the biological activity and/or structure of the polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a polypeptide that correspond to amino acid residues that are important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of that information, one skilled in the art may predict the alignment of amino acid residues with respect to its three dimensional structure. One skilled in the art may choose not to make radical changes to amino acid residues predicted to be on the surface of the protein, since such residues may be involved in important interactions with other molecules. Moreover, one skilled in the art may generate test derivatives containing a single amino acid substitution at each desired amino acid residue. The derivatives can then be screened using activity assays known to those skilled in the art and as disclosed herein. Such derivatives could be used to gather information about suitable substitution. For example, if one discovered that a change to a particular amino acid residue resulted in destroyed, undesirably reduced, or unsuitable activity, derivatives with such a change would be avoided. In other words, based on information gathered from such routine experiments, one skilled in the art can readily determine the amino acids where further substitutions should be avoided either alone or in combination with other mutations.

Numerous scientific publications have been devoted to the prediction of secondary structure from analyses of amino acid sequences (see Chou et al., Biochemistry, 13(2):222-245, 1974; Chou et al., Biochemistry, 113(2):211-222, 1974; Chou et al., Adv. Enzymol. Relat. Areas Mol. Biol., 47:45-148, 1978; Chou et al., Ann. Rev. Biochem., 47:251-276, 1979; and Chou et al., Biophys. J., 26:367-384, 1979). Moreover, computer programs are currently available to assist with predicting secondary structure of polypeptides. Examples include those programs based upon the Jameson-Wolf analysis (Jameson et al., Comput. Appl. Biosci., 4(1):181-186, 1998; and Wolf et al., Comput. Appl. Biosci., 4(1):187-191; 1988), the program PepPlot® (Brutlag et al., CABS, 6:237-245, 1990; and Weinberger et al., Science, 228:740-742, 1985), and other new programs for protein tertiary structure prediction (Fetrow et al., Biotechnology, 11:479-483, 1993).

Moreover, computer programs are currently available to assist with predicting secondary structure. One method of predicting secondary structure is based upon homology modeling. For example, two polypeptides or proteins which have a sequence identity of greater than 30%, or similarity greater than 40%, often have similar structural topologies. The recent growth of the protein structural data base (PDB) has provided enhanced predictability of secondary structure, including the potential number of folds within a polypeptide's or protein's structure (see Holm et al., Nucl. Acid. Res., 27(1):244-247, 1999).

Additional methods of predicting secondary structure include “threading” (Jones, D., Curr. Opin. Struct. Biol., 7(3):377-87, 1997; Suppl et al., Structure, 4(1):15-9, 1996), “profile analysis” (Bowie et al., Science, 253:164-170, 1991; Gribskov et al., Meth. Enzym., 183:146-159, 1990; and Gribskov et al., Proc. Nat. Acad. Sci., 84(13): 4355-4358, 1987), and “evolutionary linkage” (See Home, supra, and Brenner, supra).

### C. Methods of Measuring Risk of Atherosclerotic Disease

Methods of measuring a subject's risk of atherosclerotic disease are provided. In general embodiment, the method comprises performing any of the above methods of measuring functional PCSK9 in a sample from the subject; and determining the subject to be at elevated or reduced risk of atherosclerotic disease based on the amount of functional PCSK9 detected.

The method may comprise determining the subject to be at elevated risk of atherosclerotic disease if the amount of functional PCSK9 in the sample is above a threshold level. In an alternative embodiment of the method, the method comprises determining the subject to be at reduced risk of atherosclerotic disease if the amount of functional PCSK9 in the sample is below a threshold level. The threshold level may be a measure of central tendency based on typical levels observed in one or more populations. For example, the threshold level may be a mean level of the functional PCSK9 observed in a given population. The given population may be defined by one or more of geography, age, ethnicity, sex, and medical history. The threshold level may take into account a measure of variation combined with a measure of central tendency. For example, the threshold level may be a mean level of the functional PCSK9 observed in a given population, plus or minus a margin of error.

The threshold level may be based on past measurements of functional PCSK9 in the subject. In such embodiments of the method the threshold level could simply be an increase or decrease in functional PCSK9 of a certain amount, or it could be a calculated rate of increase or decrease in functional PCSK9. The threshold level will in some cases be a trend as opposed to an absolute amount.

Some embodiments of the method comprise measuring the total circulating concentration of PCSK9 in the subject. This can then be compared to the total measured functional PCSK9 in the subject. In such embodiments the subject's risk may be calculated as a function of functional PCSK9 and total circulating PCSK9. One example of such a function is a ratio of functional PCSK9 to total circulating PCSK9. Total circulating PCSK9 can be calculated by any means known in the art, for example by ELISA or other immunological approaches.

The method may further employ the use of any of the apparatuses or kits described below.

### D. Apparatus and Kit for Measuring Functional PCSK9

An apparatus for measuring functional PCSK9 in a sample is provided. A general embodiment of the apparatus comprises: a substrate 100 with low binding affinity to PCSK9; and a binding agent 200 associated with the substrate 100. The binding agent 200 may be any that is disclosed in the preceding sections as suitable for use in the method. In some embodiments of the method the binding agent 200 is immobilized on the substrate 200.

The substrate 100 is a surface that has a relatively low tendency to bind or retain PCSK9. The substrate 100 may be a structure that is commonly used in biotechnological applications, such as a sample dish, a multi-well plate, a bead, a filter, or a microsphere. Such biotechnological substrates 100 are typically constructed from glass or polymers such as polystyrene. They may be coated to increase hydrophobicity or to facilitate the immobilization of the PCSK9-binding agent 200. The substrate 100 may be blackened to increase the sensitivity of fluorescent and luminescent reporters. The substrate 100 may also be transparent or translucent to enable the detection of fluorescent, luminescent, and colorimetric reporters from different angles. Some embodiments of the substrate 100 are constructed of non-fluorescent material, which has the advantage of introducing no interference when a fluorescent reporter is measured.

In a particular embodiment the substrate 100 is a bead that contains a scintillant, sometimes referred to as a “fluoromicrosphere.” Such fluoromicrospheres have the advantage of allowing very sensitive detection of radiolabeled substrate, such as when the reporter is a radioisotope. Fluoromicrospheres may be of any size, but commonly they are submicroscopic (0.2-15 μm). In an alternative embodiment the substrate is a polystyrene culture plate.

Some embodiments of the apparatus comprise a sample in contact with the binding peptide, the sample comprising PCSK9 300. The sample may be of any type that is disclosed as suitable for the method in the preceding sections. Some embodiments of the apparatus comprise a signal compound 400 in contact with the binding agent 200, the signal compound 400 comprising a LDL-R-binding region capable of binding to the binding agent 200 that is associated with the apparatus, and a reporter 500. Again, the signal compound 400 may be of any type that is disclosed as suitable for the method in the preceding sections, and will have the property of binding to the binding agent 200.

A kit is provided for evaluating a subject's risk of atherosclerotic disease. In a general embodiment the kit comprises any of the apparatuses disclosed above; and a signal compound 400, the signal compound comprising a LDL-R-binding region capable of binding to the binding agent that is associated with the apparatus, and a reporter 500. The signal compound 400 in the kit has the property of binding to the PCSK9-binding region 200 that is associated with the apparatus. The signal compound 400 may be any that is disclosed as suitable for use in the method or the apparatus above.

The kit may further comprise one or more standard solutions of PCSK9 that can be used to calibrate the assay. Some embodiments of the kit may comprise a plurality of standard solutions of PCSK9; such embodiments have the advantage of providing standards that can be used to develop a standard curve that is accurate over a range of concentrations. In a specific embodiment the kit comprises standard solutions of PCSK9 ranging from about 1.5 to about 100 ng mL⁻¹ PCSK9. FIG. 6 illustrates a working example of a standard curve using seven standard solutions ranging from about 1.5-100 ng mL⁻¹ PCSK9.

### E. Fret Reagents

A fluorescence resonance energy transfer (FRET) reagent for the detection of functional PCSK9 is provided, comprising a PCSK9-binding agent conjugated to a first fluorophore. The PCSK9-binding agent may be any that is disclosed above. The fluorophore may be any donor or acceptor fluorophore, which are generally known to those of ordinary skill in the art.

A kit for FRET detection of PCSK9 is provided comprising the FRET reagent above; and a second FRET reagent comprising: a signal compound, said signal compound comprising a LDL-R-binding region capable of binding to the binding agent, and a second fluorophore that is complementary fluorophore to the first fluorophore. The LDL-R-binding region may be any that is disclosed as suitable in the embodiments described above.

In this context a “complementary fluorophore” is a donor fluorophore capable of electron transfer to stimulate the emission of a given acceptor fluorophore if the first fluorophore is an acceptor fluorophore; or an acceptor fluorophore capable of emission in response to electron transfer from a given donor fluorophore if the first fluorophore is a donor fluorophore.

### F. Working Example

A quantitative assay for the detection of functional PCSK9 in circulation was developed and validated. A recombinant EGF-AB domain (of the LDL-R) for the capture of circulating functional PCSK9 and the biotinylated conjugate of the His(×6)-tagged PCSK9 protein (serve as a competitor) formed the basis for this non-antibody assay.

The linear detection range of the assay showed a correlation coefficient of R²>0.99. The assay was able to quantitate free functional PCSK9 in circulation, had a detection limit of 1.89 ng/mL, and a limit of quantitation of 5.32 ng/mL. The ratio (free PCSK9/total PCSK9) displayed strong positive correlation with free functional PCSK9 levels (r=0.77, P<0.00001, N=50), while it showed a negative correlation with total PCSK9 (r=−0.56, P<0.00001, N=50).

The functional PCSK9 assay and the derived information provide informative and actionable data for the proper dosing of PCSK9 antagonists, monitoring of treatment efficacy and CVD risk assessment.

1. Materials and Methods

a. Sample Handling and Testing

Routine blood samples were collected in 10-mL lavender-top EDTA tubes using standard phlebotomy practices. Immediately after collection, tubes were gently inverted five times, and then centrifuged at 2,500 rpm for 15 min at 4° C. The supernatant plasma was transferred into 2 ml cryogenic vials and frozen at −80° C. until analysis. VAP cholesterol panel tests were performed at Atherotech Diagnostics Laboratory according to lab SOP. Total plasma PCSK9 concentrations were measured in duplicate, using CircuLex Human PCSK9 ELISA kit (Cyclex, Nagano, Japan), as per the manufacturer's instructions. To ascertain the stability of PCSK9 in frozen plasma/sera, samples subjected to no more than 3 freeze-thaw cycles were selected for analysis.

b. Free PCSK9 Assay Development and Validation

An assay for the detection of free functional PCSK9 in circulation that is capable of binding to LDL-R was modified from CircuLex PCSK9-LDL-R in vitro binding assay kit (Cyclex, Nagano, Japan). The method was developed and optimized for plasma and serum samples, its performance characteristics were established and validated. Calibrators were prepared using serial dilution of recombinant His(×6)-tagged PCSK9 proteins to establish 7-point standards of 100, 50, 25, 12.5, 6.25, 3.125, 1.56 ng/mL. Plasma/serum samples were used straight without dilution and added directly to wells that had been pre-coated with the recombinant LDL-R-EGF-AB domain. In parallel, a bovine serum albumin (BSA) solution (final concentration 12.5-25 mg/mL) was added to the standard wells. The plate was then incubated at room temperature for 2 hrs. After four washes in an ELx50 programmable automatic plate washer (BioTek, Winooski, Vt.), a saturated amount of 100 ng/mL His(×6)-tagged PCSK9 (to bind any remaining free recombinant LDL-R-EGF-AB) was added to each sample well whereas serially diluted calibrator His(×6)-tagged PCSK9 were added to standard wells, then incubated for 1 hr at room temperature. All consecutive washing steps performed between each procedure in this assay were the same. After another 1-hr incubation with a 1:150 biotinylated anti-His-tag monoclonal antibody and a 20-min incubation with a 1:150 streptavidin-HRP (horseradish peroxidase) conjugate, the chromogenic substrate reagent (tetra-methylbenzidine, TMB) was added and the signal was detected at 450 nm and quantified in BioTek Synergy HT microplate reader using Gen5 software (BioTek, Winooski, Vt.). The concentration of His(×6)-tagged PCSK9 bound in each sample well was generated based on the 7-point standard curve, and the free functional PCSK9 in each plasma sample can be calculated using the formula:

Free functional PCSK9 (ng/mL)=100 (ng/mL)−His(×6)-tagged PCSK9 bound (ng/mL) Eq. 1

The validation of the free functional PCSK9 competitive ELISA generally followed the IUPAC (International Union of Pure and Applied Chemistry) guidelines and recommendations.

c. Determining Linearity Based on PCSK9 Level

High, medium, and low PCSK9 samples were pooled from 3-4 previously tested patient samples. The corresponding samples were collected and categorized based on their total or functional PCSK9 protein levels:

High-level: >500 ng/mL for total PCSK9, and >40 ng/mL for free PCSK9;
Medium-level: 200-500 ng/mL for total PCSK9, and 20-40 ng/mL for free PCSK9;
Low-level: <200 ng/mL for total PCSK9, and <20 ng/mL for free PCSK9.

d. Statistical Analysis

Linear regression analysis was applied to evaluate the association between total PCSK9, functional PCSK9, ratio. of functional over total PCSK9 and patient characteristics, LDL-C, or total cholesterol, and to assess differences in plasma PCSK9 levels among high, medium, and low risk groups. A P-value<0.05 was considered statistically significant.

2. Results

The assay in this example was effective to measure circulating levels of functional PCSK9 that has the ability to bind to LDL-R using the recombinant EGF-AB domain as bait. His(×6)-tagged PCSK9 was used as a competitor for the endogenous PCSK9.

a. Linearity

The detection range of the assay was defined as the linear part of the curve with a correlation coefficient of R²>0.99 when linear regression was applied. The linearity of the sample and the standard were determined by assaying the samples at different dilutions and the His(×6)-tagged PCSK9 calibrator at various concentrations, and determining the dynamic detection range. A linear standard curve was established with the His(×6)-tagged PCSK9 with serial 1:2 dilutions. The correlation coefficient R² of the His(×6)-tagged PCSK9 calibrators tested ranging from 6.25 to 100 ng/mL was 0.994-0.998 (FIGS. 6(a)-(d)). The standard curve of total PCSK9 showed a correlation coefficient R²=0.999-1.000.

The method provided linear results for samples binned by PCSK9 level (high, medium, low) for both functional and total PCSK9 (see FIGS. 7A and 7B).

b. Sensitivity

The limit of detection (LOD) of the free functional PCSK9 assay was calculated as the mean of the measured concentrations of 11 buffer blank samples plus 3 times the standard deviation (SD) of the mean value. The limit of quantitation (LOQ) was calculated as the mean of the measured concentrations of 11 buffer blank samples plus 10 times the standard deviation (SD) of the mean value. The assay had a detection limit of 0.34 ng/mL, and a LOQ of 1.04 ng/mL (Table 5).

TABLE 5
LOD and LOQ for free functional PCSK9 ELISA

| Blank Samples | Conc. (ng/mL) |
|---|---|
| 1 | 0 |
| 2 | 0 |
| 3 | 0 |
| 4 | 0 |
| 5 | 0 |
| 6 | 0 |
| 7 | 0 |
| 8 | 0 |
| 9 | 0.089 |
| 10 | 0.32 |
| Mean | 0.04 |
| SD | 0.10 |
| Mean + 3 SD | 0.34 |
| Mean + 10 SD | 1.04 |

The LOD and LOQ of total PCSK9 assay were also determined on 10 buffer blank samples to be 0.059 and 0.178 ng/mL, respectively (Table 6).

TABLE 6
LOD and LOQ for total steady-state PCSK9 ELISA

| Blank Samples | Conc. (ng/mL) |
|---|---|
| 1 | 0 |
| 2 | 0 |
| 3 | 0 |
| 4 | 0 |
| 5 | 0 |
| 6 | 0 |
| 7 | 0 |
| 8 | 0 |
| 9 | 0.033 |
| 10 | 0.046 |
| Mean | 0.0079 |
| SD | 0.017 |
| Mean + 3 SD | 0.059 |
| Mean + 10 SD | 0.178 |

c. Clinical validation

Plasma from 50 clinical samples was collected and tested on both the free functional PCSK9 assay and total PCSK9 assay. The mean of total PCSK9, free functional PCSK9, and percent ratio (free/total) was 369.44±158.32 ng/mL, 29.47±15.65 ng/mL, and 9.54±6.64, respectively. That the numbers of total and free PCSK9 were generally in line with other reports (26, 27) further demonstrated the accuracy of the system, although the “free” PCSK9 in most literature refers to a PCSK9 pool that is available to be targeted by therapeutic humanized antibodies and not the “functional PCSK9” measured by the method here.

Plasma levels of steady-state PCSK9 were higher in women than in men (394 vs. 307 ng/mL), again consistent with other reports and may reflect an increase in the synthesis of PCSK9 or reduced clearance of the protein from the circulation in women.

3. Reference Range

A reference range was initially determined by assaying 15-16 apparently healthy normal adult plasma samples, and calculated using Mean±1.96SD to cover 95% confidence interval. The reference ranges for plasma samples are 54.73-760.73 ng/mL for total PCSK9, and 40.53-57.11 ng/ml for functional PCSK9 (Tables 8 & 9). A comparison of values of total and functional PCSK9 levels in human plasma and serum was completed using 20 matched samples from normal adult samples (see Tables 10 & 11). Overall, although total PCSK9 concentrations were comparable in serum and plasma samples, functional PCSK9 values were higher and more consistent in serum than those from plasma.

4. Variation

The intra-assay variation is defined as the reproducibility of a sample within an assay and was generated from assaying 9 replicates from each the three QC pools. The coefficient of variation (CV) for 3 replicates of a sample pool was used to determine if the reproducibility is acceptable 15% CV except for the LOQ level QC, where the acceptability is ≦20%). Statistics performed on the results determined that the reproducibility (CV) for the three QC pools for total PCSK9 were 2.90, 2.62 and 2.12% with mean concentrations of 174.91, 306.22 and 495.87 ng/mL respectively (see Table 14). The reproducibility for three pools for functional PCSK9 were 21.34, 4.25 and 4.12%, with mean concentrations of 14.48, 33.43, and 39.60 ng/ml respectively (Table 12).

The inter-assay variation is defined as the reproducibility (CV) of a sample between assays. Using the three QC Pools covering the reportable range of the assay, evaluated over 3 runs, the inter-assay variation (CV) for the pools was determined to be 6.00, 6.74 and 8.14%, with mean concentrations of 182.44, 301.33 and 461.20 ng/mL respectively for total PCSK9 (Table 15), and 11.99, 10.75, 2.63% with mean concentrations of 34.53, 35.99 and 38.04 ng/ml (Table 13). All pools met the acceptable reproducibility requirements of ≦15% CV.

### G. Supported Embodiments

The following embodiments are specifically contemplated and disclosed in this application so as to support claims currently or as necessary in later applications that may cite the benefit of the instant application.

### Embodiment 1

A method of measuring functional proprotein convertase subtilisin-like/kexin type 9 (PCSK9) in a sample, the method comprising:
(a) contacting the sample with a PCSK9-binding agent capable of binding to the LDL-R-binding region of a PCSK9 for a period sufficient to allow substantially all of the PCSK9 in the sample to bind to the binding agent; and
(b) measuring directly or indirectly the amount of functional PCSK9 from the sample bound to the binding agent.

### Embodiment 2

Any of the above in which the step of measuring directly or indirectly the amount of functional PCSK9 from the sample bound to the binding agent comprises: (a) contacting the binding agent with a signal compound capable of binding to the binding agent, the signal compound comprising a reporter; and (b) measuring the amount of signal compound bound to the binding agent.

### Embodiment 3

Any of the above comprising removing any unbound signal compound.

### Embodiment 4

Any of the above in which LDL has not been removed from the sample.

### Embodiment 5

Any of the above comprising removing LDL from the sample.

### Embodiment 6

Any of the above comprising removing free LDL from the sample.

### Embodiment 7

Any of the above wherein an excess of binding agent is present compared to the expected PCSK9 in the sample.

### Embodiment 8

Any of the above wherein the sample is a supernatant produced by centrifugation.

### Embodiment 9

Any of the above comprising centrifuging an aliquot of blood to remove substantially all of the LDL and to produce a supernatant, and wherein the supernatant is the sample.

### Embodiment 10

Any of the above wherein the sample is blood plasma.

### Embodiment 11

Any of the above comprising measuring the total PCSK9 in the sample.

### Embodiment 12

Any of the above wherein the sample is from a subject, further comprising measuring the total circulating concentration of PCSK9 in the subject.

### Embodiment 13

Any of the above wherein the sample is from a subject, further comprising measuring the total PCSK9 in the sample in addition to the functional PCSK9.

### Embodiment 14

Any of the above when performed ex vivo.

### Embodiment 15

Any of the above when performed in vitro.

### Embodiment 16

A use of a PCSK9-binding agent capable of binding to the LDL-R-binding region of a PCSK9 for the measurement of functional PCSK9 in a sample.

### Embodiment 17

The use of embodiment 16 in combination with the use of a signal compound capable of binding to the binding agent, the signal compound comprising a reporter.

### Embodiment 18

A use of a PCSK9-binding agent capable of binding to the LDL-R-binding region of a PCSK9 for the evaluation of a subject's risk of atherosclerotic disease.

### Embodiment 19

The use of embodiment 18 in combination with the use of a signal compound capable of binding to the binding agent, the signal compound comprising a reporter.

### Embodiment 20

A diagnostic method of evaluating a subject's risk of atherosclerotic disease, the method comprising: performing any one of the methods of embodiments 1-15 on a plasma sample from the subject; and determining the subject's risk of atherosclerotic disease based on the amount of functional PCSK9 measured.

### Embodiment 21

The diagnostic method of embodiment 20, in which the step of measuring directly or indirectly the amount of functional PCSK9 from the sample bound to the binding agent comprises: (a) contacting the binding agent with a signal compound capable of binding to the binding agent, the signal compound comprising a reporter; and (b) measuring the amount of signal compound bound to the binding agent.

### Embodiment 22

Any one of the diagnostic methods of embodiments 20-21, comprising determining the subject to be at elevated risk of atherosclerotic disease if the amount of functional PCSK9 in the sample is below a threshold level.

### Embodiment 23

Any one of the diagnostic methods of embodiments 20-22, comprising determining the subject to be at reduced risk of atherosclerotic disease if the amount of functional PCSK9 in the sample is above a threshold level.

### Embodiment 24

Any one of the diagnostic methods of embodiments 20-23, further comprising measuring the total concentration of circulating of PCSK9 in the subject.

### Embodiment 25

Any one of the diagnostic methods of embodiments 20-24, comprising determining the subject's risk of atherosclerotic disease based on a ratio of functional PCSK9 to total circulating PCSK9 in the subject.

### Embodiment 26

Any one of the diagnostic methods of embodiments 20-25, when performed ex vivo.

### Embodiment 27

Any one of the diagnostic methods of embodiments 20-26, when performed in vitro.

### Embodiment 28

An apparatus for measuring functional PCSK9 in a sample, the apparatus comprising: a substrate with low binding affinity to PCSK9; and a PCSK9-binding agent associated with the substrate, said binding agent capable of binding to the LDL-R-binding region of a PCSK9.

### Embodiment 29

The apparatus of embodiment 28, wherein the PCSK9-binding agent is immobilized on the substrate.

### Embodiment 30

The apparatus of any one of embodiments 28-29, wherein the substrate is selected from the group consisting of: a sample dish, a multi-well plate, a bead, and a microsphere.

### Embodiment 31

The apparatus of any one of embodiments 28-30, wherein the substrate is non-fluorescent.

### Embodiment 32

The apparatus of any one of embodiments 28-31, wherein the substrate comprises a scintillant.

### Embodiment 33

The apparatus of any one of embodiments 28-32, wherein the substrate is translucent.

### Embodiment 34

The apparatus of any one of embodiments 28-33, wherein the substrate is transparent.

### Embodiment 35

The apparatus of any one of embodiments 28-34, comprising a sample in contact with the binding agent, the sample comprising PCSK9.

### Embodiment 36

The apparatus of any one of embodiments 28-35, comprising a signal compound in contact with the binding agent, the signal compound comprising a LDL-R-binding region and a reporter.

### Embodiment 37

A method of measuring functional PCSK9 in a sample, the method comprising contacting the sample with the apparatus of any one of embodiments 28-36 for a period sufficient to allow substantially all of the PCSK9 in the sample to bind to the binding agent; and measuring directly or indirectly the amount of functional PCSK9 from the sample bound to the binding agent.

### Embodiment 38

The method of embodiment 37 in which the step of measuring directly or indirectly the amount of functional PCSK9 from the sample bound to the binding agent comprises: (a) contacting the binding agent with a signal compound capable of binding to the binding agent, the signal compound comprising a reporter; and (b) measuring the amount of signal compound bound to the binding agent.

### Embodiment 39

A kit for of evaluating a subject's risk of atherosclerotic disease, the kit comprising: the apparatus of any one of embodiments 28-36; and a signal compound capable of binding to the binding agent, the signal compound comprising a reporter.

### Embodiment 40

A fluorescence resonance energy transfer (FRET) reagent for the detection of functional PCSK9, comprising a PCSK9-binding agent conjugated to a first fluorophore.

### Embodiment 41

A kit for fluorescence resonance energy transfer (FRET) detection of PCSK9, comprising: a FRET reagent for the detection of functional PCSK9, comprising a PCSK9-binding agent conjugated to a first fluorophore; and a second FRET reagent comprising: a signal compound capable of binding to the binding agent, and a second fluorophore that is a complementary fluorophore to the first fluorophore.

### Embodiment 42

Any one of the above embodiments comprising a signal compound, wherein the signal compound comprises an LDL-R-binding region.

### Embodiment 43

Any one of the above embodiments comprising a signal compound, wherein the signal compound comprises a LDL-R-binding region of a PCSK9.

### Embodiment 44

Any one of the above embodiments comprising a signal compound, wherein the signal compound comprises a catalytic domain of a PCSK9 polypeptide.

### Embodiment 45

Any one of the above embodiments comprising a signal compound, wherein the signal compound comprises a peptide sequence having at least a certain level of homology to SEQ ID NO: 14, said homology selected from the group consisting of: 75, 80, 85, 90, 95, 99, 99.9, and 100%.

### Embodiment 46

Any one of the above embodiments comprising a signal compound, wherein the signal compound comprises a peptide sequence having at least a certain level of homology to SEQ ID NO: 14, said homology selected from the group consisting of: 75, 80, 85, 90, 95, 99, 99.9, and 100%, and wherein the residues at positions 157, 174, 215, 216, 218, 219, 237, 239, 253, 357, 374, 391, 394, 417, 425, 443, and 452 are selected from Table 2.

### Embodiment 47

Any one of the above embodiments comprising a signal compound, wherein the signal compound comprises a peptide sequence having at least a certain level of homology to SEQ ID NO: 13, said homology selected from the group consisting of: 75, 80, 85, 90, 95, 99, 99.9, and 100%.

### Embodiment 48

Any one of the above embodiments comprising a signal compound, wherein the signal compound comprises a peptide sequence having at least a certain level of homology to SEQ ID NO: 12, said homology selected from the group consisting of: 75, 80, 85, 90, 95, 99, 99.9, and 100%.

### Embodiment 49

Any one of the above embodiments comprising a signal compound, wherein the signal compound comprises a peptide sequence having the following structure:

R₁-R₂-R₃-R₄-R₅

in which:
R₁ is a sequence with a minimum level of homology to SEQ ID NO: 27, said minimum level of homology selected from: 75, 80, 85, 90, 95, 97, 98, 99, 99.5, and 100%;
R₂ is an oligopeptide of 0-5 residues;
R₃ is a sequence with a minimum level of homology to SEQ ID NO: 28, said minimum level of homology selected from: 75, 80, 85, 90, 95, 97, 98, 99, 99.5, and 100%;
R₄ is a peptide of 0-1 residues; and
R₅ is a sequence with a minimum level of homology to positions 31-691 SEQ ID NO: 22 limited to variants in which each of positions 31, 47, 86, 92, 166, 171, 205, 301, 381, 433, 448, 505, 530, 554, 573, 574, 591, 662, 665, and 685 are independently selected from C, D, E, H, K, N, Q, R, S, or T; each of positions 41, 82, 87, 108, 118, 161, 164, 200, 246, 296, 379, 407, 416, 439, 452, 455, 469, 498, 499, 511, 577, 622, 642, and 652, are independently selected from A, C, F, G, H, I, K, L, M, R, T, V, W, or Y; each of positions 50, 67, 127, 176, 190, 245, 544, 616, 648, and 661 are independently selected from A, G, or S; each of positions 52, 55, 58, 59, 70, 99, 163, 167, 172, 191, 206, 244, 279, 298, 306, 312, 395, 400, 419, 442, 450, 473, 531, 537, 538, 540, 555, 569, 572, 580, 585, 615, 635, 636, 640, 643, 655, 660, 663, 664, 671, 672, 674, and 684 are independently selected from A, C, D, G, N, P, S, T, and V; each of positions 53, 69, 140, 168, 402, 479, and 497 are independently selected from D or E; each of 54, 56, 116, 131, 401, 404, 449, 542, 546, 571, 582, 592, 618, 627, 668, and 669 are independently selected from A, C, D, E, G, H, K, N, Q, R, S, or T; each of positions 64, 95, 247, 302, 493, 509, 601, and 658 are independently selected from H, K, or R; each of positions 78, 107, 110, 113, 201, 276, 295, 522, 428, 535, 595, and 609 are independently selected from I, L, or V; each of positions 115, 378, and 596 are independently selected from F, H, W, or Y; each of positions 174 and 365 are independently selected from D, E, H, K, or R; and said minimum level of homology selected from: 75, 80, 85, 90, 95, 97, 98, 99, 99.5, and 100%.

### Embodiment 50

Any one of the above embodiments comprising a signal compound, wherein the signal compound comprises a peptide sequence having at least a certain level of homology to SEQ ID NO: 23, said homology selected from the group consisting of: 75, 80, 85, 90, 95, 99, 99.9, and 100%.

### Embodiment 51

Any one of the above embodiments comprising a signal compound, wherein the signal compound comprises a peptide sequence having at least a certain level of homology to SEQ ID NO: 23, said homology selected from the group consisting of: 75, 80, 85, 90, 95, 99, 99.9, and 100%, wherein the residues at positions 10, 12-17, 19-21, 23, 25, 27, 39, 40, 49, 50, 54, 55, 93-96, 125, 128, 144, 145, 147, 150, 151, 155, 161, 214, 227, 228, 230, 244, 246, 249-251, 253, 265, 268, 274, 282, 288, 291, and 297 are selected from FIG. 2.

### Embodiment 52

Any one of the above embodiments comprising a signal compound, wherein the signal compound comprises a peptide sequence having at least a certain level of homology to the catalytic domain at least one of: SEQ ID NO: 17, SEQ ID NO: 18, SEQ ID NO: 19, and SEQ ID NO: 20; said homology selected from the group consisting of: 75, 80, 85, 90, 95, 99, 99.9, and 100%.

### Embodiment 53

Any one of the above embodiments comprising a signal compound, wherein the reporter is selected from the group consisting of: a fluorescent moiety, a luminescent moiety, a radionuclide, a magnetic particle, a dye, a donor fluorophore, an acceptor fluorophore, and an enzyme.

### Embodiment 54

Any one of the above embodiments comprising a signal compound, wherein the reporter is a first fluorophore, and the binding agent is conjugated to second fluorophore that is a complementary fluorophore to the first fluorophore.

### Embodiment 55

Any one of the above embodiments comprising a signal compound, wherein the signal compound comprises a peptide sequence comprising SEQ ID NO: 13.

### Embodiment 56

Any one of the above embodiments, wherein the PCSK9-binding agent comprises a PCSK9-binding region.

### Embodiment 57

Any one of the above embodiments, wherein the PCSK9-binding agent comprises a PCSK9-binding region of a low-density lipoprotein receptor (LDL-R).

### Embodiment 58

Any one of the above embodiments, wherein the binding agent comprises an EGF-AB domain of the LDL-R.

### Embodiment 59

Any one of the above embodiments, wherein the binding agent comprises an EGF-A domain of the LDL-R.

### Embodiment 60

Any one of the above embodiments, wherein the binding agent comprises the N-terminal region of an EGF-A domain of the LDL-R.

### Embodiment 61

Any one of the above embodiments, wherein the binding agent comprises a peptide sequence having at least a certain level of homology to positions 1-26 of SEQ ID NO: 11 said homology selected from the group consisting of: 75, 80, 85, 90, 95, 99, 99.9, and 100%.

### Embodiment 62

Any one of the above embodiments, wherein the binding agent comprises a peptide sequence having at least a certain level of homology to positions 1-40 of SEQ ID NO: 11 said homology selected from the group consisting of: 75, 80, 85, 90, 95, 99, 99.9, and 100%.

### Embodiment 63

Any one of the above embodiments, wherein the binding agent comprises a peptide sequence having at least a certain level of homology to SEQ ID NO: 11 said homology selected from the group consisting of: 75, 80, 85, 90, 95, 99, 99.9, and 100%.

### Embodiment 64

Any one of the above embodiments, wherein the binding agent comprises a peptide sequence having at least a certain level of homology to positions 1-26 of SEQ ID NO: 10, said homology selected from the group consisting of: 75, 80, 85, 90, 95, 99, 99.9, and 100%.

### Embodiment 65

Any one of the above embodiments, wherein the binding agent comprises a peptide sequence having at least a certain level of homology to positions 1-40 of SEQ ID NO: 10, said homology selected from the group consisting of: 75, 80, 85, 90, 95, 99, 99.9, and 100%.

### Embodiment 66

Any one of the above embodiments, wherein the binding agent comprises a peptide sequence having at least a certain level of homology to SEQ ID NO: 10, said homology selected from the group consisting of: 75, 80, 85, 90, 95, 99, 99.9, and 100%.

### Embodiment 67

Any one of the above embodiments, wherein the binding agent comprises a peptide sequence having at least a certain level of homology with positions 1-26 of SEQ ID NO: 8, said homology selected from the group consisting of: 75, 80, 85, 90, 95, 99, 99.9, and 100%.

### Embodiment 68

Any one of the above embodiments, wherein the binding agent comprises a peptide sequence having at least a certain level of homology with SEQ ID NO: 8, said homology selected from the group consisting of: 75, 80, 85, 90, 95, 99, 99.9, and 100%.

### Embodiment 69

Any one of the above embodiments, wherein the binding agent comprises a peptide sequence having at least a certain level of homology with SEQ ID NO: 8, wherein the residues at positions 318, 327, 329, 335, 338, 350, 352, 354, 356, 357, 368, 370, 379, and 391 are selected from Table 1; said homology selected from the group consisting of: 75, 80, 85, 90, 95, 99, 99.9, and 100%.

### Embodiment 70

Any one of the above embodiments, wherein the binding agent comprises a peptide sequence having at least a certain level of homology with SEQ ID NO: 9; said homology selected from the group consisting of: 75, 80, 85, 90, 95, 99, 99.9, and 100%.

### Embodiment 71

Any one of the above embodiments, wherein the binding agent comprises a peptide sequence having at least a certain level of homology with SEQ ID NO: 9, wherein the residues at positions 318, 327, 329, 335, 338, 350, 352, 354, 356, 357, 368, 370, 379, and 391 are selected from Table 1; said homology selected from the group consisting of: 75, 80, 85, 90, 95, 99, 99.9, and 100%.

### Embodiment 72

Any one of the above embodiments, wherein the binding agent comprises a peptide sequence having at least a certain level of homology with positions 1-26 of SEQ ID NO: 24; said homology selected from the group consisting of: 75, 80, 85, 90, 95, 99, 99.9, and 100%.

### Embodiment 73

Any one of the above embodiments, wherein the binding agent comprises a peptide sequence having at least a certain level of homology with SEQ ID NO: 24; said homology selected from the group consisting of: 75, 80, 85, 90, 95, 99, 99.9, and 100%.

### Embodiment 74

Any one of the above embodiments, wherein the binding agent comprises a peptide sequence having at least a certain level of homology with SEQ ID NO: 25, said homology selected from the group consisting of: 75, 80, 85, 90, 95, 99, 99.9, and 100%.

### Embodiment 75

Any one of the above embodiments, wherein the binding agent comprises a peptide sequence having at least a certain level of homology with positions 314-339 of at least one of: SEQ ID NO: 1, SEQ ID NO: 2, SEQ ID NO: 3, SEQ ID NO: 15, and SEQ ID NO: 16; said homology selected from the group consisting of: 75, 80, 85, 90, 95, 99, 99.9, and 100%.

### Embodiment 76

Any one of the above embodiments, wherein the binding agent comprises a peptide sequence having at least a certain level of homology with positions 314-353 of at least one of: SEQ ID NO: 1, SEQ ID NO: 2, SEQ ID NO: 3, SEQ ID NO: 15, and SEQ ID NO: 16; said homology selected from the group consisting of: 75, 80, 85, 90, 95, 99, 99.9, and 100%.

### Embodiment 77

Any one of the above embodiments, wherein the binding agent comprises a peptide sequence having at least a certain level of homology with positions 314-393 of at least one of: SEQ ID NO: 1, SEQ ID NO: 2, SEQ ID NO: 3, SEQ ID NO: 15, and SEQ ID NO: 16; said homology selected from the group consisting of: 75, 80, 85, 90, 95, 99, 99.9, and 100%.

### Embodiment 78

Any one of the above embodiments, wherein the binding agent comprises a peptide sequence having at least a certain level of homology with positions 314-339 of at SEQ ID NO: 1, said homology selected from the group consisting of: 75, 80, 85, 90, 95, 99, 99.9, and 100%.

### Embodiment 79

Any one of the above embodiments, wherein the binding agent comprises a peptide sequence having at least a certain level of homology with positions 314-353 of at SEQ ID NO: 1, said homology selected from the group consisting of: 75, 80, 85, 90, 95, 99, 99.9, and 100%.

### Embodiment 80

Any one of the above embodiments, wherein the binding agent comprises a peptide sequence having at least a certain level of homology with positions 314-393 of SEQ ID NO: 1, said homology selected from the group consisting of: 75, 80, 85, 90, 95, 99, 99.9, and 100%.

### Embodiment 81

Any one of the above embodiments, wherein the binding agent comprises a peptide sequence comprising positions 314-339 of SEQ ID NO: 1.

### Embodiment 82

Any one of the above embodiments, wherein the binding agent is conjugated to a fluorophore.

### H. Conclusions

It is to be understood that any given elements of the disclosed embodiments of the invention may be embodied in a single structure, a single step, a single substance, or the like. Similarly, a given element of the disclosed embodiments may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and are capable of changes or modifications within the scope of the teachings as expressed herein, commensurate with the skill and/or knowledge of a person having ordinary skill in the relevant art. The embodiments: described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. §1.77 or otherwise to provide organizational queues. These headings shall not limit or characterize the invention(s) set forth herein.

TABLE 7
Key to Sequence Listing

| | |
|---|---|
| SEQ ID NO: 1 | Human LDL-R Isoform 1 |
| SEQ ID NO: 2 | Pan troglodytes LDL-R |
| SEQ ID NO: 3 | Macaca mulatta LDL-R |
| SEQ ID NO: 4 | Human LDL-R Isoform 2 |
| SEQ ID NO: 5 | Human LDL-R Isoform 3 |
| SEQ ID NO: 6 | Human LDL-R Isoform 4 |
| SEQ ID NO: 7 | Human LDL-R Isoform 5 |
| SEQ ID NO: 8 | Human LDL-R Isoform 1 PCSK9 Binding Domain |
| | at Positions 314-353 (including natural variants) |
| SEQ ID NO: 9 | Human LDL-R Isoform 1 PCSK9 Binding Domain |
| | at Positions 314-393 (including natural variants) |
| SEQ ID NO: 10 | Primate Consensus PCSK9 Binding Domain of LDL-R |
| SEQ ID NO: 11 | Mammalian Consensus PCSK9 Binding Domain of |
| | LDL-R |
| SEQ ID NO: 12 | Human PCSK9 Isoform 1 |
| SEQ ID NO: 13 | Human PCSK9 Isoform 1 Catalytic Domain |
| SEQ ID NO: 14 | Human PCSK9 Isoform 1 Catalytic Domain (including |
| | natural variants) |
| SEQ ID NO: 15 | Mus musculus LDL-R |
| SEQ ID NO: 16 | Rattus norvegicus LDL-R |
| SEQ ID NO: 17 | Pan troglodytes PCSK9 |
| SEQ ID NO: 18 | Macaca mulatta PCSK9 |
| SEQ ID NO: 19 | Mus musculus PCSK9 |
| SEQ ID NO: 20 | Rattus norvegicus PCKS9 |
| SEQ ID NO: 21 | Primate Consensus PCSK9 |
| SEQ ID NO: 22 | Mammalian Consensus PCSK9 |
| SEQ ID NO: 23 | Mammalian Consensus PCSK9 Catalytic Domain |
| SEQ ID NO: 24 | Human LDL-R Isoform 1 positions 314-353 allowing |
| | for any substitution at the positions shown |
| | in Table 1 |
| SEQ ID NO: 25 | Human LDL-R Isoform 1 positions 314-393 allowing |
| | for any substitution at the positions shown |
| | in Table 1 |
| SEQ ID NO: 26 | Functional Mammalian Consensus PCSK9 Binding |
| | Domain of LDL-R |
| SEQ ID NO: 27 | Mammalian Consensus PCSK9 N-Terminal Sequence |
| SEQ ID NO: 28 | Mammalian Consensus PCSK9 Central Sequence |

TABLE 9
Reference range for free functional PCSK9 in plasma

| Samples | Conc. (ng/mL) |
|---|---|
| 1 | 43.85 |
| 2 | 48.43 |
| 3 | 47.91 |
| 4 | 51.32 |
| 5 | 51.76 |
| 6 | 49.40 |
| 7 | 49.43 |
| 8 | 54.69 |
| 9 | 47.34 |
| 10 | 49.75 |
| 11 | 54.93 |
| 12 | 42.48 |
| 13 | 48.55 |
| 14 | 55.18 |
| 15 | 41.36 |
| 16 | 44.81 |
| Mean | 48.82 |
| SD | 4.23 |
| Median | 48.98 |
| Range | 41.36-55.18 |

TABLE 10
Total PCSK9 concentrations in 20 matched plasma and
serum samples

| | Plasma conc. | Serum conc. |
|---|---|---|
| Samples | (ng/mL) | (ng/mL) |
| 1 | 429.00 | 329.60 |
| 2 | 239.80 | 367.40 |
| 3 | 330.20 | 421.40 |
| 4 | 449.80 | 463.40 |
| 5 | 479.00 | 556.00 |
| 6 | 545.20 | 697.80 |
| 7 | 279.60 | 320.20 |
| 8 | 612.80 | 872.20 |
| 9 | 449.80 | 335.60 |
| 10 | 262.60 | 295.80 |
| 11 | 434.40 | 689.00 |
| 12 | 465.40 | 352.60 |
| 13 | 358.00 | 289.00 |
| 14 | 314.00 | 348.60 |
| 15 | 378.20 | 355.20 |
| 16 | 255.20 | 245.20 |
| 17 | 406.00 | 291.00 |
| 18 | 335.00 | 317.40 |
| 19 | 317.40 | 274.80 |
| 20 | 239.00 | 251.20 |
| Mean | 379.02 | 403.67 |
| SD | 104.27 | 170.27 |
| Median | 358.00 | 335.60 |

TABLE 11
Free functional PCSK9 concentrations in 20 matched plasma and
serum samples

| | Plasma conc. | Serum conc. |
|---|---|---|
| Samples | (ng/mL) | (ng/mL) |
| 1 | 62.46 | 89.87 |
| 2 | 57.25 | 87.16 |
| 3 | 56.88 | 85.70 |
| 4 | 49.64 | 84.30 |
| 5 | 61.63 | 90.76 |
| 6 | 75.18 | 94.93 |
| 7 | 70.95 | 93.83 |
| 8 | 71.63 | 94.15 |
| 9 | 84.87 | 91.02 |
| 10 | 73.61 | 92.95 |
| 11 | 26.00 | 92.32 |
| 12 | 62.15 | 88.73 |
| 13 | 51.05 | 91.02 |
| 14 | 58.55 | 92.27 |
| 15 | 68.92 | 93.42 |
| 16 | 60.58 | 93.52 |
| 17 | 80.60 | 73.77 |
| 18 | 85.50 | 69.86 |
| 19 | 76.89 | 72.62 |
| 20 | 70.90 | 72.62 |
| Mean | 65.26 | 87.24 |
| SD | 13.90 | 8.22 |
| Median | 65.69 | 90.80 |

TABLE 12
Intra-assay variation for free functional PCSK9 ELISA

| Conc. | Replicate | | | | | |
|---|---|---|---|---|---|---|
| (ng/mL) | 1 | Replicate 2 | Replicate 3 | Mean | SD | % CV |
| Free | 12.12 | 17.99 | 13.35 | 14.48 | 3.09 | 21.34 |
| PCSK9 | | | | | | |
| (L) | | | | | | |
| Free | 33.12 | 34.97 | 32.19 | 33.43 | 1.42 | 4.25 |
| PCSK9 | | | | | | |
| (M) | | | | | | |
| Free | 41.46 | 38.37 | 38.99 | 39.60 | 1.63 | 4.12 |
| PCSK9 | | | | | | |
| (H) | | | | | | |

TABLE 13
Inter-assay variation for free functional PCSK9 ELISA

| Conc. (ng/mL) | Day 1 | Day 2 | Day 3 | Mean | SD | % CV |
|---|---|---|---|---|---|---|
| Free PCSK9 (L) | 39.32 | 32.17 | 32.12 | 34.53 | 4.14 | 11.99 |
| Free PCSK9 (M) | 35.84 | 39.93 | 32.19 | 35.99 | 3.87 | 10.75 |
| Free PCSK9 (H) | 37.00 | 38.12 | 38.99 | 38.04 | 1.00 | 2.63 |

TABLE 14
Intra-assay variation for Total PCSK9 ELISA

| Conc. | Replicate | Replicate | | | | |
|---|---|---|---|---|---|---|
| (ng/mL) | 1 | 2 | Replicate 3 | Mean | SD | % CV |
| Total | 176.00 | 169.40 | 179.33 | 174.91 | 5.06 | 2.90 |
| PCSK9 | | | | | | |
| (L) | | | | | | |
| Total | 297.33 | 308.40 | 312.93 | 306.22 | 8.02 | 2.62 |
| PCSK9 | | | | | | |
| (M) | | | | | | |
| Total | 505.20 | 497.93 | 484.47 | 495.87 | 10.52 | 2.12 |
| PCSK9 | | | | | | |
| (H) | | | | | | |

TABLE 15
Inter-assay variation for Total PCSK9 ELISA

| Conc. (ng/mL) | Day 1 | Day 2 | Day 3 | Mean | SD | % CV |
|---|---|---|---|---|---|---|
| Total PCSK9 (L) | 194.60 | 173.40 | 179.33 | 182.44 | 10.94 | 6.00 |
| Total PCSK9 (M) | 313.20 | 277.87 | 312.93 | 301.33 | 20.32 | 6.74 |
| Total PCSK9 (H) | 417.87 | 481.27 | 484.47 | 461.20 | 37.56 | 8.14 |

I claim:

1. A method of evaluating a subject's risk of atherosclerotic disease by selectively measuring functional proprotein convertase subtilisin-like/kexin type 9 (PCSK9) in a sample containing an unknown amount of PCKS9, the method comprising:
(a) contacting the sample with a PCSK9-binding agent, said binding agent comprising a first peptide sequence from the N-terminal region of the PCSK9 binding domain of a low-density lipoprotein receptor, for a period sufficient to allow substantially all of the PCSK9 in the sample to bind to the binding agent;
(b) contacting the binding agent with a signal compound, the signal compound comprising: (i) a reporter, and (ii) a second peptide sequence from the catalytic domain of a PCSK9;
(c) measuring the amount of signal compound bound to the binding agent; and
(d) determining the subject's risk of atherosclerotic disease based on the amount of functional PCSK9 measured.

2. The method of claim 1 comprising removing any unbound signal compound.

3. The method of claim 1 further comprising measuring the total PCSK9 in the sample in addition to the functional PCSK9.

4. The method of claim 1 further comprising removing free LDL from the sample.

5. The method of claim 1, in which the first peptide sequence has at least 90% identity with positions 1-26 of at least one sequence selected from the group consisting of: SEQ ID NO: 26, SEQ ID NO: 10; SEQ ID NO: 9 or SEQ ID NO: 25.

6. The method of claim 1, in which the first peptide sequence has at least 90% identity with positions 314-339 of at least one sequence selected from the group consisting of: SEQ ID NO: 1, SEQ ID NO: 2, SEQ ID NO: 3, SEQ ID NO:15, and SEQ ID NO: 16.

7. The method of claim 1, in which the first peptide sequence has at least 90% identity with positions 1-40 of at least one sequence selected from the group consisting of: SEQ ID NO: 26, SEQ ID NO: 10; SEQ ID NO: 9 or SEQ ID NO: 25.

8. The method of claim 1, in which the first peptide sequence has at least 90% identity with positions 314-353 of at least one sequence selected from the group consisting of: SEQ ID NO: 26, SEQ ID NO: 10; SEQ ID NO: 9 or SEQ ID NO: 25 SEQ ID NO: 1, SEQ ID NO: 2, SEQ ID NO: 3, SEQ ID NO:15 and SEQ ID NO: 16.

9. The method of claim 1, in which the first peptide sequence has at least 90% identity with positions 1-80 of at least one sequence selected from the group consisting of: SEQ ID NO: 26, SEQ ID NO: 10; SEQ ID NO: 9 or SEQ ID NO: 25.

10. The method of claim 1, in which the first peptide sequence has at least 90% identity with positions 314-393 of at least one sequence selected from the group consisting of: SEQ ID NO: 1, SEQ ID NO: 2, SEQ ID NO: 3, SEQ ID NO:15, and SEQ ID NO: 16. The method of claim 1, in which the second peptide sequence has at least 90% identity with SEQ ID NO: 23, SEQ ID NO: 14 or SEQ ID NO: 13.

11. The method of claim 1, in which the first peptide sequence has at least 95% identity with SEQ ID NO: 26 and in which the second peptide sequence has at least 95% identity with SEQ ID NO: 23.

12. An apparatus for measuring functional PCSK9 in a sample, the apparatus comprising: a substrate with low binding affinity to PCSK9; and a PCSK9-binding agent associated with the substrate, wherein said binding agent comprises a first peptide sequence from the N-terminal region of the PCSK9 binding domain of a low-density lipoprotein receptor, and wherein an excess of binding agent is present compared to the expected PCSK9 in the sample.

13. The apparatus of claim 13, in which the first peptide sequence has at least 90% identity with positions 1-26 of at least one sequence selected from the group consisting of: SEQ ID NO: 26, SEQ ID NO: 10; SEQ ID NO: 9 or SEQ ID NO: 25.

14. The apparatus of claim 13, in which the first peptide sequence has at least 90% identity with positions 314-339 of at least one sequence selected from the group consisting of: SEQ ID NO: 1, SEQ ID NO: 2, SEQ ID NO: 3, SEQ ID NO:15, and SEQ ID NO: 16.

15. The apparatus of claim 13, in which the first peptide sequence has at least 90% identity with positions 1-40 of at least one sequence selected from the group consisting of: SEQ ID NO: 26, SEQ ID NO: 10; SEQ ID NO: 9 or SEQ ID NO: 25.

16. The apparatus of claim 13, in which the first peptide sequence has at least 90% identity with positions 314-353 of at least one sequence selected from the group consisting of: SEQ ID NO: 26, SEQ ID NO: 10; SEQ ID NO: 9 or SEQ ID NO: 25 SEQ ID NO: 1, SEQ ID NO: 2, SEQ ID NO: 3, SEQ ID NO:15 and SEQ ID NO: 16.

17. The apparatus of claim 13, in which the first peptide sequence has at least 90% identity with positions 1-80 of at least one sequence selected from the group consisting of: SEQ ID NO: 26, SEQ ID NO: 10; SEQ ID NO: 9 or SEQ ID NO: 25.

19. The apparatus of claim 13, in which the first peptide sequence has at least 90% identity with positions 314-393 of at least one sequence selected from the group consisting of: SEQ ID NO: 1, SEQ ID NO: 2, SEQ ID NO: 3, SEQ ID NO:15, and SEQ ID NO: 16.

20. A kit for fluorescence resonance energy transfer (FRET) detection of functional PCSK9 in a sample, comprising: a FRET reagent for the detection of functional PCSK9, comprising a PCSK9-binding agent conjugated to a first fluorophore, said binding agent comprising a first peptide sequence from the N-terminal region of the PCSK9 binding domain of a low-density lipoprotein receptor; and a second FRET reagent comprising: a second fluorophore that is a complementary fluorophore to the first fluorophore, and a signal compound capable of binding to the binding agent, said binding agent comprising a second peptide sequence from the catalytic domain of a PCSK9; wherein an excess of binding agent is present compared to the expected PCSK9 in the sample.


SEQUENCE LISTING (28 sequences)

SEQ ID NO: 1 - protein, 860 aa, Homo sapiens [sequence omitted]

SEQ ID NO: 2 - protein, 860 aa, Pan troglodytes
MEPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQHCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQHPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTVLEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPNTSRLPGATPGLTTVEIVTTSHQALGDVAGRGNEKKPSSVRALSIILPIVLLVFLCLGAFLLWKNWRLKNINSINFDNPVYQKTTEDEVHICRNQDGYSYPSRQMVSLEDDVA

SEQ ID NO: 3 - protein, 853 aa, Macaca mulatta
MGPWGWKLRWTVAFLLAAAEAAVGDRCERNEFQCEDGKCISYKWVCDGTAECQDGSDESQETCLSVTCKSGDFSCGGRVNRCIPQFWRCDGEVDCENGSDEQDCPPKTCSQDEFRCHDGKCIYRQFVCDSDRDCLDGSDEASCPVLTCGPASFQCNSSTCIPQLWACDNDPDCEDGSDEWPQHCQGLEVPKRDSSPCSAFEFHCRSGECIHSGWRCDGGPDCKDKSDEENCPEFQCSDGTCIHGSRQCDREYDCKDMSDEVGCINVTLCEGPNKFKCHSGECISLDKVCNMARDCRDWSDEPIKECGTNECLDNNGGCSHICNDLKIGYECLCPDGFQLVAQRRCEDIDECQDPDTCSQLCVNLEGSYKCQCEEGFQLDPHTKACKAVGSIAYLIFTNRHEVRKMTLDRSEYTSLIPNLRNVVALDTEVASNRIYWSDLSQRMIYSTQLDRAHSVSSYDTVISRDLQAPDGLAVDWIHSNIYWTDSVLGTVSVADTKGVKRKTLFRENGSKPRAIVVDPVHGFMYWTDWGTPAKIKKGGLNGVDIYSLVTENIEWPNGITLDFPSGRLYWVDSKLHSISSIDVNGGNRKTILEDKERLAHPFSLAIFEDKVFWTDIINEAIFSANRLTGSDINLLAENLLSPEDMVLFHNLTQPRGVNWCERTTLSNGGCQYLCLPAPQINPQSPKFTCTCPDGMLLAKDMRSCLTEAEAAVATQETSTVRLMVSSKAVATQHTTTRPVPNTSQLPGATPGLTTAETVTMSHQALGDVAGRGNEKKPKSVGALSIVLPTVLLVFLCLGAFLLWKNWRLKSINSINFDNPVYQKTTEDEVHICRNQDGYSYPSRQMVSLEDDVA

SEQ ID NO: 4 - protein, 682 aa, Homo Sapiens
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPREAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 5 - protein, 693 aa, Homo sapiens
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPLTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 6 - protein, 819 aa, Homo sapiens
MGPWGWKLRWTVALLLAAAGTAVGDRCERNEFQCQDGKCISYKWVCDGSAECQDGSDESQETCSPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 7 - protein, 858 aa, Homo sapiens
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSMVSLEDDVA

SEQ ID NO: 8 - protein, 40 aa, Homo sapiens
  Variant (5)..(5): Xaa= C, F, R, or Y
GTNEXLDNNGGCSXVXNDLKIXYEXLCPXXFQLVAQXRXE

SEQ ID NO: 9 - protein, 80 aa, Homo sapiens
  variant (5)..(5): Xaa = C, F, R, or Y
GTNEXLDNNGGCSXVXNDLKIXYEXLCPXXFQLVAQXRXEXIXXXQDPDTXSQLXVXLEGGYKCQXEEGFQLDPHTKXCK

SEQ ID NO: 10 - protein, 80 aa, Artificial Sequence
  Consensus between Homo sapiens, Pan troglodytes, and Macaca mulatta
GTNECLDNNGGCSHXCNDLKIGYECLCPDGFQLVAQRRCEDIDECQXPDTCSQLCVNLEGXYKCQCEEGFQLDPHTKACK

SEQ ID NO: 11 - protein, 80 aa, Artificial Sequence
  Consensus between Homo sapiens, Pan troglodytes, Macaca mulatta, Mus musculus, and Rattus norvegicus
GTNECLDNNGGCSHXCXDLKIGXECLCPXGFXLVXXXRCEDIDECQXPDTCSQLCVNLEGXYKCXCXXGFXXDPHTXXCK

SEQ ID NO: 12 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 13 - protein, 297 aa, Homo sapiens
SIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTH

SEQ ID NO: 14 - protein, 297 aa, Homo sapiens
  variant (5)..(5): Xaa = N or K
SIPWXLERITPPRYRADEYQPXDGGSLVEVYLLDTSIQSDHREIEGRVMVTDFENVPEEDGTXXHXXASKCDSHGTHLAGVVSGXDXGVAKGASMRSLRVXNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGXCVDLFAPGEDIIGASSXCSTCFVSQSGTSQAAAXVAXIAAMMLSAEPELTLAELRQRLIXFSAKDVIXEAWFPEDQRVLTPNLVAXLPPSTH

SEQ ID NO: 15 - protein, 862 aa, Mus musculus
MSTADLMRRWVIALLLAAAGVAAEDSCSRNEFQCRDGKCIASKWVCDGSPECPDGSDESPETCMSVTCQSNQFSCGGRVSRCIPDSWRCDGQVDCENDSDEQGCPPKTCSQDDFRCQDGKCISPQFVCDGDRDCLDGSDEAHCQATTCGPAHFRCNSSICIPSLWACDGDVDCVDGSDEWPQNCQGRDTASKGVSSPCSSLEFHCGSSECIHRSWVCDGEADCKDKSDEEHCAVATCRPDEFQCADGSCIHGSRQCDREHDCKDMSDELGCVNVTQCDGPNKFKCHSGECISLDKVCDSARDCQDWSDEPIKECKTNECLDNNGGCSHICKDLKIGSECLCPSGFRLVDLHRCEDIDECQEPDTCSQLCVNLEGSYKCECQAGFHMDPHTRVCKAVGSIGYLLFTNRHEVRKMTLDRSEYTSLLPNLKNVVALDTEVTNNRIYWSDLSQKKIYSALMDQAPNLSYDTIISEDLHAPDGLAVDWIHRNIYWTDSVPGSVSVADTKGVKRRTLFQEAGSRPRAIVVDPVHGFMYWTDWGTPAKIKKGGLNGVDIHSLVTENIQWPNGITLDLSSGRLYWVDSKLHSISSIDVNGGNRKTILEDENRLAHPFSLAIYEDKVYWTDVINEAIFSANRLTGSDVNLVAENLLSPEDIVLFHKVTQPRGVNWCETTALLPNGGCQYLCLPAPQIGPHSPKFTCACPDGMLLAKDMRSCLTEVDTVLTTQGTSAVRPVVTASATRPPKHSEDLSAPSTPRQPVDTPGLSTVASVTVSHQVQGDMAGRGNEEQPHGMRFLSIFFPIALVALLVLGAVLLWRNWRLKNINSINFDNPVYQKTTEDELHICRSQDGYTYPSRQMVSLEDDVA

SEQ ID NO: 16 - protein, 879 aa, Rattus norvegicus
MSTADLMLRWAIALLLAAAGVAAEDSCGKNEFQCRDGKCIVSKWVCDGSRECPDGSDESPETCMSVTCRSGEFSCGGRVSRCIPDSWRCDGRTDCENGSDELDCSPKTCSLDEFRCQDGKCISRQFVCDQDWDCLDGSDEAHCAATTCGPAHFRCNSSSCIPSLWACDGDRDCDDGSDEWPQNCGAEDTAAEVVSSPCSSLEFHCGSSECIHRSWVCDGAADCKDKSDEENCAVTTCRPDEFQCADGSCIHGSRQCDREHDCKDMSDELGCINVTQCDGPNKFKCHSGECISLDKVCNSARDCRDWSDEPIKECKTNECLDNNGGCSHICKDLKIGYECLCPSGFRLVDGHQCEDIDECQEPDTCSQLCVNLEGSFKCECRAGFHMDPHTRVCKAVGSIGFLLFTNRHEVRKMTLDRSEYTSLIPNLKNVVALDTEVANNRIYWSDLSQRKIYSAVMDQGTSLSYDAIISGDLHAPDGLAVDWIHGNIYWTDSVPGTVSVADTKGVRRRTLFREKGSRPRAIVVDPVHGFMYWTDWGTPAKIKKGGLNGVDIYSLVTEDIQWPNGITLDLPSGRLYWVDSKLHSISSIDVNGGGRKTILEDEKQLAHPFSLAIYEDKVYWTDVLNEAIFSANRLTGSDVNLVAKNLMSPEDIVLFHNVTQPRGVNWCEATVLPNGGCQYMCLPAPQISAHSPKFTCACPDGMLLAKDMRSCLPEVDTVPTTQGTSTIGPVVTTSAAVSLKRKEDPSATRHKEDPSATRHNEDPSATSTSRQPGDTPELSTVESVTVSSQVQGDMAGRGDEVQRHGVGFLSIFLPIALVALLVFGAILLWRNWRLRNINSINFDNPVYQKTTEDEIHICRSQDGYTYPSRQMVSLEDDVA

SEQ ID NO: 17 - protein, 692 aa, Pan troglodytes
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSIHTAPPAEAGMGTRVHCHQQGHVLTGCSSHWEVEDLGTHKPPMLRPRGQPNQCVGHREASIHASCCRAPGLECKVKEHGIPAPQEQVTVACEEGWTLTGCSALPGTSHVLGAYAVDNTCVVRSRDVSTAGSTSEEAVAAVAICCRSRHLAQASQELQ

SEQ ID NO: 18 - protein, 692 aa, Macaca mulatta
MGTVSSRRSWWPLPLPLLLLLLLGPAGARAQEDEDGDYEELVLALRSEEDGLADAPEHGATATFHRCAKDPWRLPGTYVVVLKEETHRSQSERTARRLQAQAARRGYLTKILHVFHHLLPGFLVKMSGDLLELALKLPHVDYIEEDSSVFAQSIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAGWQLFCRTVWSAHSGPTRMATAVARCAQDEELLSCSSFSRSGKRRGERIEAQGGKRVCRAHNAFGGEGVYAIARCCLLPQVNCSVHTAPPAGASMGTRVHCHQQGHVLTGCSSHWEVEDLGTHKPPVLRPRGQPNQCVGHREASIHASCCHAPGLECKVKEHGIPAPQEQVIVACEDGWTLTGCSPLPGTSHVLGAYAVDNTCVVRSRDVSTTGSTSKEAVAAVAICCRSRHLVQASQELQ

SEQ ID NO: 19 - protein, 694 aa, Mus musculus
MGTHCSAWLRWPLLPLLPPLLLLLLLLCPTGAGAQDEDGDYEELMLALPSQEDGLADEAAHVATATFRRCSKEAWRLPGTYIVVLMEETQRLQIEQTAHRLQTRAARRGYVIKVLHIFYDLFPGFLVKMSSDLLGLALKLPHVEYIEEDSFVFAQSIPWNLERIIPAWHQTEEDRSPDGSSQVEVYLLDTSIQGAHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNAACRHLARTGVVLVAAAGNFRDDACLYSPASAPEVITVGATNAQDQPVTLGTLGTNFGRCVDLFAPGKDIIGASSDCSTCFMSQSGTSQAAAHVAGIVARMLSREPTLTLAELRQRLIHFSTKDVINMAWFPEDQQVLTPNLVATLPPSTHETGGQLLCRTVWSAHSGPTRTATATARCAPEEELLSCSSFSRSGRRRGDWIEAIGGQQVCKALNAFGGEGVYAVARCCLVPRANCSIHNTPAARAGLETHVHCHQKDHVLTGCSFHWEVEDLSVRRQPALRSRRQPGQCVGHQAASVYASCCHAPGLECKIKEHGISGPSEQVTVACEAGWTLTGCNVLPGASLTLGAYSVDNLCVARVHDTARADRTSGEATVAAAICCRSRPSAKASWVQ

SEQ ID NO: 20 - protein, 691 aa, Rattus norvegicus
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 21 - protein, 692 aa, Artificial Sequence
  Consensus between Homo sapiens, Pan troglodytes, and Macaca mulatta
MGTVSSRRSWWPLPLXLLLLLLLGPAGARAQEDEDGDYEELVLALRSEEDGLAXAPEHGXTATFHRCAKDPWRLPGTYVVVLKEETHXSQSERTARRLQAQAARRGYLTKILHVFHXLLPGFLVKMSGDLLELALKLPHVDYIEEDSSVFAQSIPWNLERITPXRYRADEYQPPXGGSLVEVYLLDTSIQSDHREIEGRVMVTDFEXVPEEDGTRFHRQASKCDSHGTHLAGVVSGRDAGVAKGAXXRSLRVLNCQGKGTVSGTLIGLEFIRKSQLVQPVGPLVVLLPLAGGYSRVXNAACQRLARAGVVLVTAAGNFRDDACLYSPASAPEVITVGATNAQDQPVTLGTLGTNFGRCVDLFAPGEDIIGASSDCSTCFVSXSGTSQAAAHVAGIAAMMLSAEPELTLAELRQRLIHFSAKDVINEAWFPEDQRVLTPNLVAALPPSTHXAGWQLFCRTVWSAHSGPTRMATAVARCAXDEELLSCSSFSRSGKRRGERXEAQGGKXVCRAHNAFGGEGVYAIARCCLLPQXNCSXHTAPPAXAXMGTRVHCHQQGHVLTGCSSHWEVEDLGTHKPPXLRPRGQPNQCVGHREASIHASCCXAPGLECKVKEHGIPAPQEQVXVACEXGWTLTGCSXLPGTSHVLGAYAVDNTCVVRSRDVSTXGSTSXXAVXAVAICCRSRHLXQASQELQ

SEQ ID NO: 22 - protein, 691 aa, Artificial Sequence
  Consensus between Homo sapiens, Pan troglodytes, Macaca mulatta, Mus musculus, and Rattus norvegicus.
MGXXXSXXXXWPLXXXLLLLLLLXPXGXXAXDEDGDYEELXLALXSXEDXLXXXXXHXXTATFXRCXKXXWRLPGTYXVVLXEETXXXQXEXTAXRLQXXAARRGYXXKXLHXFXXLXPGFLVKMSXDLLXLALKLPHVXYIEEDSXVFAQSIPWNLERIXPXXXXXXEXXXPXGXSXVEVYLLDTSIQXXHREIEGRVXXTDFXXVPEEDGTRFHRQASKCDSHGTHLAGVVSGRDAGVAKGXXXXSLRVLNCQGKGTVSGTLIGLEFIRKSQLXQPXGPLVVLLPLAGGYSRXXNXACXXLARXGVVLVXAAGNFRDDACLYSPASAPEVITVGATNAQDQPVTLGTLGTNFGRCVDLFAPGXDIIGASSDCSTCXXSXSGTSQAAAHVAGIXAXMLXXXPXLTLAELRQRLIXFSXKDVINXAWFPEDQXVLTPNXVAXLPPSTXXXGXQLXCRTVWSAHSGPTRXATAXARCAXXEELLSCSSFSRSGXRRGXXXEAXGGXXVCXAXNAFGGEGVYAXARCCLXPXXNCSXHXXPXAXAXXXTXVHCHQXXHVLTGCSXHWEVEXLXXXXXPXLRXRXQPXQCVGHXXASXXASCCXAPGLECKXKEHGIXXPXEQVXVACEXGWTLTGCXXLPGXSXXLGAYXVDNXCVXRXXDXXXXXXTSXXAXXAXAICCRSRXXXXASXXXX

SEQ ID NO: 23 - protein, 297 aa, Artificial Sequence
  Consensus between Homo sapiens, Pan troglodytes, Macaca mulatta, Mus musculus, and Rattus norvegicus.
SIPWNLERIXPXXXXXXEXXXPXGXSXVEVYLLDTSIQXXHREIEGRVXXTDFXXVPEEDGTRFHRQASKCDSHGTHLAGVVSGRDAGVAKGXXXXSLRVLNCQGKGTVSGTLIGLEFIRKSQLXQPXGPLVVLLPLAGGYSRXXNXACXXLARXGVVLVXAAGNFRDDACLYSPASAPEVITVGATNAQDQPVTLGTLGTNFGRCVDLFAPGXDIIGASSDCSTCXXSXSGTSQAAAHVAGIXAXMLXXXPXLTLAELRQRLIXFSXKDVINXAWFPEDQXVLTPNXVAXLPPSTX

SEQ ID NO: 24 - protein, 40 aa, Homo sapiens; identical to SEQ ID NO: 8
  misc_feature (5)..(5): Xaa can be any naturally occurring amino acid

SEQ ID NO: 25 - protein, 80 aa, Homo sapiens; identical to SEQ ID NO: 9
  misc_feature (5)..(5): Xaa can be any naturally occurring amino acid

SEQ ID NO: 26 - protein, 80 aa, Artificial Sequence
  LDLR PCSK9 binding domain mammalian consensus sequence
XTNECLDNNGGCSHXCXDLKIGXECLCPXGFXLVXXXXCEDIDECQXPDTCSQLCVNLEGXXKCXCXXGFXXDPHTXXCK

SEQ ID NO: 27 - protein, 14 aa, Artificial Sequence
  PCSK9 Mammalian Consensus Sequence N-Terminal Sequence
MGXXXSXXXXWPLX

SEQ ID NO: 28 - protein, 16 aa, artificial sequence
  Consensus sequence
XXLLLLLLLXPXGXXA